SEQUENCE LISTING (57 sequences)

SEQ ID NO: 1 - DNA, 34 bp, Escherichia coli
gcatttagctaccgggcagtgccattttcgaaat

SEQ ID NO: 2 - DNA, 95 bp, Escherichia coli
  misc_feature (69)..(70): octanediol group connects residues 69 and 70
tcgtcttccggtctctcctctcaagcctcagcgctctctctccctatagtgagtcgtattaatttcgaaggcatgacaacccgaacaccagtgat

SEQ ID NO: 3 - DNA, 21 bp, Escherichia coli
  misc_feature (21)..(21): 3′ PCR-block
gcgtccactccggtcctctcg

SEQ ID NO: 4 - DNA, 30 bp, Escherichia coli
  misc_feature (30)..(30): 3′ PCR block
gcttagatgctttcagcacttatctcttcc

SEQ ID NO: 5 - DNA, 111 bp, Escherichia coli
  misc_feature (76)..(77): octanediol group connects residues 76 and 77
tgcctgcttgtctgcgttctggatatcacccgagttctcgcttcctatagtgagtcgtattaatttctcgtcttccggtctctcctctcaagcctcagcgctctctctccc

SEQ ID NO: 6 - DNA, 12 bp, Escherichia coli
  misc_feature (1)..(1): 5′ alkaline phosphatase labelled
ggatatcacccg

SEQ ID NO: 7 - DNA, 25 bp, Escherichia coli
  misc_feature (1)..(1): 5′ biotin labelled
tctgctgcctgcttgtctgcgttct

SEQ ID NO: 8 - DNA, 34 bp, Escherichia coli; identical to SEQ ID NO: 1

SEQ ID NO: 9 - DNA, 95 bp, Escherichia coli; identical to SEQ ID NO: 2
  misc_feature (69)..(70): octanediol group connects residues 69 and 70

SEQ ID NO: 10 - DNA, 21 bp, Escherichia coli
  misc_feature (21)..(21): 3′ PCR block
gcgtccactccggtcctctcg

SEQ ID NO: 11 - DNA, 30 bp, Escherichia coli; identical to SEQ ID NO: 4
  misc_feature (30)..(30): 3′ PCR block

SEQ ID NO: 12 - DNA, 111 bp, Escherichia coli; identical to SEQ ID NO: 5
  misc_feature (76)..(77): octanediol group connects residues 76 and 77

SEQ ID NO: 13 - DNA, 12 bp, Escherichia coli
  misc_feature (12)..(12): 3′ horse radish peroxidase labelled
ggatatcacccg

SEQ ID NO: 14 - DNA, 25 bp, Escherichia coli
  misc_feature (1)..(1): 5′ biotin labelled
tctgctgcctgcttgtctgcgttct

SEQ ID NO: 15 - DNA, 34 bp, Escherichia coli; identical to SEQ ID NO: 1

SEQ ID NO: 16 - DNA, 95 bp, Escherichia coli; identical to SEQ ID NO: 2
  misc_feature (69)..(70): octanediol group connects residues 69 and 70

SEQ ID NO: 17 - DNA, 44 bp, Artificial Sequence
  DNA homologue of SMART RNA1 amplicon
gggagagagagcgctgaggcttgagaggagagaccggaagacga

SEQ ID NO: 18 - DNA, 25 bp, Artificial Sequence
  5′ biotinylated capture probe for SMART RNA1 amplicon
tctgctcgtcttccggtctctcctc

SEQ ID NO: 19 - DNA, 111 bp, Escherichia coli; identical to SEQ ID NO: 5
  misc_feature (76)..(77): octanediol group connects residues 76 and 77

SEQ ID NO: 20 - DNA, 20 bp, Artificial Sequence
  3′ DNP labelled detection probe for SMART RNA1 amplicon
gcctcagcgctctctctccc

SEQ ID NO: 21 - DNA, 45 bp, Escherichia coli
ggaagcgagaactcgggtgatatccagaacgcagacaagcaggca

SEQ ID NO: 22 - DNA, 25 bp, Escherichia coli
  misc_feature (1)..(1): 5′ biotin labelled
tctgctgcctgcttgtctgcgttct

SEQ ID NO: 23 - DNA, 20 bp, Escherichia coli
  misc_feature (20)..(20): 3′ dinitrophenol labelled
tcacccgagttctcgcttcc

SEQ ID NO: 24 - DNA, 39 bp, Artificial Sequence
  extension probe for RNA1 generation
gcctggcaccattaaagaaaatatcatctttttcgaaat

SEQ ID NO: 25 - DNA, 111 bp, Artificial Sequence; identical to SEQ ID NO: 5
  template probe for RNA1 generation

SEQ ID NO: 26 - DNA, 121 bp, Artificial Sequence
  synthetic target for RNA1 generation
cctcctctagttggcatgctttgatgacgcttctgtatctatattcatcataggaaacaccaaagatgatattttctttaatggtgccaggcataatccaggaaaactgagaacagaatga

SEQ ID NO: 27 - DNA, 46 bp, Chlamydia trachomatis
aattctaatacgactcactatagggagcacatagactctcccttaa

SEQ ID NO: 28 - DNA, 19 bp, Chlamydia trachomatis
agcaattgtttcgacgatt

SEQ ID NO: 29 - DNA, 19 bp, Chlamydia trachomatis
  misc_feature (1)..(1): 5′ biotin labelled
ggcggaagggttagtaatg

SEQ ID NO: 30 - DNA, 25 bp, Chlamydia trachomatis
  misc_feature (25)..(25): 3′ horse radish peroxidase labelled
gtggcgatatttgggcatccgagta

SEQ ID NO: 31 - DNA, 99 bp, Salmonella typhimurium
  misc_feature (69)..(70): octanediol group connects residues 69 and 70
tcgtcttccggtctctcctctcaagcctcagcgctctctctccctatagtgagtcgtattaatttcgaatccccgctgaaagtactttacaacccgaag

SEQ ID NO: 32 - DNA, 28 bp, Salmonella typhimurium
tattaaccacaacaccttccttcgaaat

SEQ ID NO: 33 - DNA, 20 bp, Salmonella typhimurium
  misc_feature (20)..(20): 3′ PCR block
gtaacgtcaattgctgcggt

SEQ ID NO: 34 - DNA, 20 bp, Salmonella typhimurium
  misc_feature (20)..(20): 3′ PCR block
gccttcttcatacacgcggc

SEQ ID NO: 35 - DNA, 85 bp, Salmonella typhimurium
  misc_feature (70)..(71): octanediol group connects residues 70 and 71
tgcctgcttgtctgcgttctggatatcacccgagctctctctccctatagtgagtcgtattaatttcgaactcctctcaagcctc

SEQ ID NO: 36 - DNA, 22 bp, Salmonella typhimurium
tcgtcttccggtctttcgaaat

SEQ ID NO: 37 - DNA, 15 bp, Salmonella typhimurium
agcgctctctctccc

SEQ ID NO: 38 - DNA, 25 bp, Salmonella typhimurium
  misc_feature (1)..(1): 5′ biotin labelled
tctgctgcctgcttgtctgcgttct

SEQ ID NO: 39 - DNA, 12 bp, Salmonella typhimurium
  misc_feature (12)..(12): 3′ horse radish peroxidase labelled
ggatatcacccg

SEQ ID NO: 40 - DNA, 99 bp, Salmonella typhimurium; identical to SEQ ID NO: 31
  misc_feature (69)..(70): octanediol group connects residues 69 and 70

SEQ ID NO: 41 - DNA, 28 bp, Salmonella typhimurium
tattaaccacaacaccttccttcgaaat

SEQ ID NO: 42 - DNA, 20 bp, Salmonella typhimurium
  misc_feature (20)..(20): 3′ PCR block
gtaacgtcaattgctgcggt

SEQ ID NO: 43 - DNA, 20 bp, Salmonella typhimurium
  misc_feature (20)..(20): 3′ PCR block
gccttcttcatacacgcggc

SEQ ID NO: 44 - DNA, 85 bp, Salmonella typhimurium; identical to SEQ ID NO: 35
  misc_feature (70)..(71): octanediol group connects residues 70 and 71

SEQ ID NO: 45 - DNA, 22 bp, Salmonella typhimurium
tcgtcttccggtctttcgaaat

SEQ ID NO: 46 - DNA, 15 bp, Salmonella typhimurium
agcgctctctctccc

SEQ ID NO: 47 - DNA, 25 bp, Salmonella typhimurium
  misc_feature (1)..(1): 5′ biotin labelled
tctgctgcctgcttgtctgcgttct

SEQ ID NO: 48 - DNA, 12 bp, Salmonella typhimurium
  misc_feature (12)..(12): 3′ horse radish peroxidase labelled
ggatatcacccg

SEQ ID NO: 49 - DNA, 99 bp, Salmonella typhimurium; identical to SEQ ID NO: 31
  misc_feature (69)..(70): octanediol group connects residues 69 and 70

SEQ ID NO: 50 - DNA, 28 bp, Salmonella typhimurium
tattaaccacaacaccttccttcgaaat

SEQ ID NO: 51 - DNA, 20 bp, Salmonella typhimurium
  misc_feature (20)..(20): 3′ PCR block
gtaacgtcaattgctgcggt

SEQ ID NO: 52 - DNA, 20 bp, Salmonella typhimurium
  misc_feature (20)..(20): 3′ PCR block
gccttcttcatacacgcggc

SEQ ID NO: 53 - DNA, 85 bp, Salmonella typhimurium; identical to SEQ ID NO: 35
  misc_feature (70)..(71): octanediol group connects residues 70 and 71

SEQ ID NO: 54 - DNA, 22 bp, Salmonella typhimurium
tcgtcttccggtctttcgaaat

SEQ ID NO: 55 - DNA, 15 bp, Salmonella typhimurium
  misc_feature (15)..(15): 3′ PCR block
agcgctctctctccc

SEQ ID NO: 56 - DNA, 25 bp, Salmonella typhimurium
  misc_feature (1)..(1): 5′ biotin labelled
tctgctgcctgcttgtctgcgttct

SEQ ID NO: 57 - DNA, 12 bp, Salmonella typhimurium
  misc_feature (12)..(12): 3′ horse radish peroxidase labelled
ggatatcacccg

(19) United States
(12) Patent Application Publication
(10) Pub. No.: US 2006/0160078 A1
(43) Pub. Date: Jul. 20, 2006

(54) Lateral flow assay device and method

(76) Inventors: Donald Leonard Nicholas Cardy, Anglesey (GB); Gerard John Allen, Surrey (GB)

Correspondence Address: MORGAN LEWIS & BOCKIUS LLP, 1111 PENNSYLVANIA AVENUE NW, WASHINGTON, DC, 20004, US

(21) Appl. No.: 10/521,111

(22) PCT Filed: Jul. 14, 2003
(86) PCT No.: PCT/GB03/03059

§ 371 Date: Sep. 23, 2005

(30) Foreign Application Priority Data
Jul. 12, 2002 (GB) 0216162.8
Sep. 4, 2002 (GB) 0220452.7

(51) Int. Cl. C12Q 1/68; C12M 1/34

(52) U.S. Cl. 435/6; 435/287.2

(57) ABSTRACT

Disclosed is a lateral flow assay device to test for the presence and/or amount of a nucleic acid sequence of interest in a sample, the lateral flow device comprising: (a) a sample receiving zone for contacting the device with a sample to be tested; (b) an extraction zone (8) for extraction of nucleic acid from the sample; (c) a nucleic acid amplification zone (18) in liquid communication with the sample receiving zone; and (d) a detection zone (20) for detecting the products, directly or indirectly, of a nucleic acid amplification reaction performed in the nucleic acid amplification zone, said detection zone (20) being, or being locatable, in liquid communication with the amplification zone (18).

### FIELD OF THE INVENTION

The present invention relates to a lateral flow assay device that may be used to detect the presence and/or amount of a target nucleic acid sequence in a sample, a kit comprising the lateral flow device, and a method of performing an assay.

### BACKGROUND OF THE INVENTION

Sensitive detection of nucleic acids has advanced over recent years with the development of a variety of nucleic acid detection and amplification techniques. These amplification techniques can be broadly divided into specific target amplification and signal amplification. Examples of target amplification techniques include, Polymerase Chain Reaction (PCR) (U.S. Pat. No. 4,683,195 and U.S. Pat. No. 4,683,202), Nucleic Acid Sequence Based Amplification (NASBA) (U.S. Pat. No. 5,130,238), and Transcription Mediated Amplification (TMA) (U.S. Pat. No. 5,399,491). Examples of signal amplification techniques include, Signal Mediated Amplification of RNA Technology (SMART) (WO 93/06240), Split Promoter Amplification Reaction (SPAR) (WO 99/37805), Invader (U.S. Pat. No. 5,846,717) and Ligase Chain Reaction (LCR) (EP 0,320,308). These technologies can be further subdivided by their ability to amplify target/signal either by requirement for thermal cycling (such as PCR) or ability to operate at a single temperature (isothermally). The content of all publications mentioned in this specification are specifically incorporated herein by reference.

One commonality between these techniques is the requirement for front end sample preparation (nucleic acid extraction) and back end amplicon detection. All of these techniques involve multi-step processes to achieve detection of the amplicon, and current development is targeted at complex systems for automation of these processes, to provide medium to high throughput of analytes.

Nucleic acid amplification techniques have been well described and illustrated in the prior art and all rely on the action of nucleic acid dependent enzymes. One such technique, SMART (WO 93/06240), relies on the interaction of two probes combined with the sequence of interest to form a three way junction (TWJ) structure generating an RNA signal after the action of DNA polymerase and an RNA polymerase. The RNA signal generated from the TWJ may be further amplified by linear amplification probes (see, for example, WO 01/09376). Detection of the RNA signal may be achieved by a number of means that are well described in the prior art that include, but are not limited to, molecular beacons (U.S. Pat. No. 5,925,517), latex beads, FRET/DFRET.

For some of the aforementioned processes, expensive, complex equipment is required together with a level of skilled labour to perform such techniques. For these techniques to become widely used for both clinical and industrial applications, reduction in the complexity of the tests (i.e. number of steps and skill base required) together with a reduction in the instrumentation and cost per test are required.

Specifically, for these tests to be applicable at the near patient (point of care or “PoC”), or near process level, simple, easy to use, cost competitive systems are required.

Chromatographic or lateral flow assays have been used for many years to simplify the performance of tests such that they can be performed by semi- or unskilled users and require minimal equipment; they are therefore ideally suited to PoC tests. To date, however, their application has primarily been restricted to immunoassays that are less complex than nucleic acid tests since they are simply detection assays, there being no amplification step.

Lateral flow tests typically utilise a single, capillary device or porous carrier that contains some (or preferably all) of the reagents necessary for the performance of an assay. These reagents are typically contained within discrete zones of the device, such that as fluid flows along the device by capillary flow the various reactions occur sequentially and a signal is generated at a detection zone that is indicative of the presence and/or amount of analyte in the sample. The devices can be a single membrane with reagents deposited at specific sites (e.g. U.S. Pat. No. 4,161,146; U.S. Pat. No. 4,361,537), or be composed of a series of discrete pads or membranes (each containing none, one or more reactants) arranged such that their edges are in liquid contact with one another (e.g. EP 0186799).

A typical lateral flow device will comprise a sample receiving zone (which may optionally contain buffers and chemicals necessary for the test), a label zone (which contains an analyte-specific binding reagent, such as an antibody, releasably bound to the membrane), a capture and detection zone (which contains an analyte-binding reagent immovably immobilized on the membrane), and an absorption zone or sink of sufficient capacity to enable unbound labelled reagent to wash out of the detection zone. The pads or membranes are typically attached to an impervious backing, and the pads or membranes are in liquid contact with one another (usually achieved by overlapping the edges and use of adhesive or a lamination layer). Optionally the device is encased in a protective housing with defined apertures for sample application and visualization of result. Examples of such devices include those disclosed in U.S. Pat. No. 5,656,503; U.S. Pat. No. 5,622,871; U.S. Pat. No. 5,602,040; U.S. Pat. No. 4,861,711.

To perform such an assay, sample is added to the sample receiving zone where it is drawn into the device by capillary force. Filter devices incorporated into the sample application zone can be used to remove blood cells, etc., and act as a volume control device (EP 0186799). The sample then hydrates and mixes with a labelled binding reagent (e.g. chromophore-labelled antibody), and any analyte present in the sample reacts with this in a specific manner to form a labelled analyte complex. This complex migrates along the device to the detection zone where a second binding reagent, immobilized on the strip, binds to the labelled analyte complex and prevents further migration of the labelled analyte complex. Unbound labelled binding reagent is drawn through the detection zone to the absorption zone. Thus, presence of signal at the detection zone is indicative of presence of analyte in the sample.

A variety of labels have been used for lateral flow assays, including radioactive (U.S. Pat. No. 4,361,537) and fluorescent labels (U.S. Pat. No. 6,238,931), although visual labels are preferred for PoC applications. These include enzyme-generated colourimetric signals (U.S. Pat. No. 4,740,468) and particulate chromophores (U.S. Pat. No. 5,591,645; U.S. Pat. No. 4,943,522; U.S. Pat. No. 5,714,389), such as colloidal gold or coloured polystyrene or “latex” particles.

Conventional immuno-based lateral flow assays use surfactants to prevent the adherence of proteins (such as antibodies and specific analytes) to the matrices used and for improvement of flow dynamic. Proteins are polymers, composed mainly of amino acids and their amino acid composition determines their electrostatic charge and hence adherence properties. In particular, proteins may have no net electrostatic charge Nucleic acids however, are polymers generally comprised of either deoxyribonucleotide or ribonucleotide units (DNA or RNA) and have a phosphate backbone that confers a net negative charge on the molecule and therefore will tend to adhere to some positively charged surfaces.

Nucleic acid-based analysis utilising lateral flow techniques has been described in the prior art. For example, WO 00/12675 discloses an assay system that integrates nucleic acid extraction, specific target amplification and detection. The device described therein comprises a hollow elongated cylinder with a single closed end and a plurality of chambers therein. Nucleic acid extraction and amplification steps occur within the cylinder. The amplified product is then contacted with the proximal end of a lateral flow test stick in order to perform the detection step of the assay. Accordingly, the device disclosed in WO 00/12675 is relatively complex. In particular, the system is not one in which the nucleic acid extraction step occurs in or on the lateral flow device.

Patent application No. US 2001/0036634 discloses an apparatus for performing a nucleic acid assay which assay involves a thermocycling amplification reaction (e.g. PCR). The apparatus comprises a lateral flow test stick and an associated thermally-regulatable apparatus whereby, as a nucleic acid amplification reaction mixture migrates along the test stick it passes through a plurality of stationary thermal zones, such that the reaction mixture is thermally cycled in a manner suitable to perform a polymerase chain reaction. The nucleic acid amplification reaction mixture is prepared outwith the assay apparatus and is applied to a sample receiving portion of the lateral flow test stick. Thus, the test stick disclosed in US 2001/0036634 does not include an integral nucleic acid extraction zone.

In addition, the arrangement disclosed in US 2001/0036634 relies on thermal cycling—this is less than ideal, especially for PoC type uses, as it requires the use of expensive thermal cycling apparatus. In addition, since PCR is a target-amplification system it is extremely sensitive to contamination. Thus a cheaper, and more robust assay device would be highly advantageous.

### SUMMARY OF THE INVENTION

In a first aspect the invention provides a lateral flow assay device to test for the presence and/or amount of a nucleic acid sequence of interest in a sample, the lateral flow device comprising:

(a) a sample receiving zone for contacting the device with a sample to be tested;

(b) an extraction zone for extraction of nucleic acid from the sample;

(c) a nucleic acid amplification zone in liquid communication with the sample receiving zone; and

(d) a detection zone for detecting the product/s, directly or indirectly, of a nucleic acid amplification reaction performed in the amplification zone, said detection zone being, or being locatable, in liquid communication with the amplification zone.

In a second aspect the invention provides a lateral flow assay device to test for the presence and/or amount of a nucleic acid sequence of interest in a sample, the lateral flow device comprising:

(a) a sample receiving zone for contacting the device with a sample to be tested;

(b) a nucleic acid isothermal amplification zone in liquid communication with the sample receiving zone; and

(c) a detection zone for detecting the product/s, directly or indirectly, of an isothermal nucleic acid amplification reaction performed in the amplification zone, said detection zone being or being locatable, in liquid communication with the amplification zone.

The assay device in accordance with the second aspect defined above differs from the assay device of the first aspect in two regards:

(i) the amplification reaction performed using the device of the first aspect may be (and preferably is) isothermal but may alternatively involve thermal cycling, whilst the amplification reaction performed using the device of the second aspect is solely isothermal;

(ii) an extraction step is performed on or within the extraction zone (typically forming part of, or adjacent to, the sample receiving zone) of the device in accordance with the first aspect—such a step may optionally also be performed (and preferably is performed) on or within the sample receiving zone of a device in accordance with the second aspect. Alternatively, however, an extraction step may be performed separately from the assay device and the resulting extracted sample subsequently applied to the sample receiving zone of a device according to the second aspect.

In a third aspect the invention provides a method of detecting the presence and/or amount of a nucleic acid sequence of interest in a sample, the method comprising the steps of: contacting a sample comprising the sequence of interest with the sample receiving zone of a lateral flow assay device in accordance with the first aspect of the invention, performing a nucleic acid extraction step on or within the lateral flow assay device, and causing a nucleic acid amplification reaction to take place in the nucleic acid amplification zone of the device; and detecting, directly or indirectly, the product/s of the amplification reaction in the detection zone of the device.

In a fourth aspect the invention provides a method of detecting the presence and/or amount of a nucleic acid sequence of interest in a sample, the method comprising the steps of: contacting a sample comprising the sequence of interest with the sample receiving zone of an assay device in accordance with the second aspect of the invention, the sample either having been subjected to an extraction step prior to contacting with the sample receiving zone or being subjected to an extraction step on or within the assay device; causing a nucleic acid amplification reaction to take place in the amplification zone of the device; and detecting, directly or indirectly, the product/s of the amplification reaction in the detection zone of the assay device.

In a fifth aspect, the invention provides a method of making a lateral flow assay device in accordance with the first and/or second aspects of the invention defined above, the method comprising the steps of: forming a porous matrix or other fluid transport means comprising an amplification zone and a detection zone said amplification zone being, or being locatable, in liquid flow communication with a sample receiving zone, the sample receiving zone comprising one or more reagents immobilised or releasably bound thereon so as to perform a nucleic acid extraction step on a nucleic-acid containing sample contacted with the sample receiving zone. Typically the one or more reagents comprise one or more (preferably all) of the following: a detergent; a base: a chelating agent; and a free radical trap. These reagents are described in greater detail elsewhere.

In a sixth aspect the invention provides an assay kit for performing an assay to test for the presence and/or amount of a nucleic acid of interest in a sample, the kit comprising a lateral flow assay device in accordance with the first and/or second aspect of the invention, and a supply of at least one reagent required to perform the assay. Conveniently the kit may additionally comprise a supply of carrier liquid, which is applied to the device during performance of the assay. The said at least one reagent may be supplied ready dissolved or suspended in the carrier liquid, or may be supplied, for example dried or lyophilised, preferably in ready-to-use aliquots. The reagent(s) supplied with the kit, separate from the assay device may be, for example, one or more of the following: a DNA polymerase; an RNA polymerase; an analyte-specific nucleic acid probe; an amplicon-specific labelling reagent; rNTPs; dNTPs and the like.

The assay device of the invention and associated aspects will now be further described. Unless the context dictates otherwise, the description below will generally apply equally to an assay device in accordance with either the first or the second aspects of the invention.

The device of the first aspect of the invention comprises, as an essential feature, a nucleic acid extraction zone. Such a zone is also a preferred feature of a device in accordance with the second aspect of the invention. The extraction zone may form a discrete portion of the device or may, for example, be comprised within the sample receiving zone. The extraction zone will typically comprise a number of reagents, one or more of which are required to perform a nucleic acid extraction step. The nucleic acid extraction reagents are conveniently localised within the extraction zone, for instance immobilised, or releasably bound (e.g. adsorbed non-specifically in dessicated form and mobilisable upon wetting). Suitable methods of achieving this are well known to those skilled in the art. The nucleic acid extraction reagents may comprise any one or more (preferably all) of the following: a detergent; a base; a chelating agent.

The “extraction” step may comprise any one of more of the following:
(i) lysis of bacterial, plant, animal, human, yeast or other fungal cells present in the sample;
(ii) disruption of any viral particles present in the sample;
(iii) at least partial purification of the nucleic acid in the sample (e.g. comprising separation of nucleic acid from fragments of lipid membrane and/or removal of polypeptide contaminants); and
(iv) inactivation of DNase and RNase present in the sample.

The amount and nature of extraction required will depend at least in part on the nature of the sample and the nature of the target sequence of interest. For example, where the analyte is double stranded a thermal (or more preferably) chemical denaturation step, typically as part of the extraction, is conveniently performed to yield single stranded nucleic acid amenable to assay. Agents suitable to cause such a chemical denaturation may conveniently be present (e.g. immobilised or releasably bound) to the extraction zone.

Alternatively, where the target is already present in single stranded form in the sample (typically, single stranded RNA) then no such thermal or chemical denaturation step will normally be required.

Once the target has been rendered single stranded, it will normally be advantageous for the target to be treated in some way to reduce the likelihood of reassociation of the strands. This could involve, for example, dilution in carrier liquid to reduce the concentration of the target strands and/or rapid contact with a considerable excess of one or more target specific probes.

In a device in accordance with the first aspect of the invention, the nucleic acid extraction is performed on or in the lateral flow device. This is also a preferred feature of a device in accordance with the second aspect of the invention. This arrangement has the advantage that essentially all the steps of the assay may be performed on the lateral flow assay device, providing greater simplicity than prior art arrangements.

The extraction step preferably occurs in and/or near the sample receiving zone. In particular, it is preferred that the sample receiving zone additionally acts as the extraction zone and comprises agents which are capable of achieving the desired extraction. Typically such agents are releasably bound or immobilized on and/or within a porous matrix. Such agents desirably include one or more (preferably all) of the following: a detergent (such as Triton X 100), a base, a chelating agent, and a free radical trap. Details of some suitable agents are contained, inter alia, within U.S. Pat. Nos. 5,496,562; 5,807,527; 5,985,327; 5,756,126; and 5,972,386.

The detergent may be an anionic detergent, such as SDS (sodium dodecyl sulphate), or non-ionic (e.g. Nonidet NP40). A particularly preferred detergent is DTAB (dodecyl trimethyl ammonium bromide), which is highly effective but readily neutralised by addition of, or contact with, cyclodextrin. Thus a DTAB/cyclodextrin system is especially suitable for the purposes of the present invention, cyclodextrin being able to neutralise the detergent which would otherwise tend to inhibit the various enzymes employed in the amplification reaction, and so require a washing step or similar to remove the DTAB prior to performing the amplification step. The DTAB/cyclodextrin system is described in further detail in WO92/12253 and U.S. Pat. No. 5,558,986. Conveniently DTAB may be provided in the nucleic acid extraction zone and cyclodextrin may be located downstream of the DTAB or else added to the assay device once the nucleic acid extraction step has been performed.

The base is advantageously a weak base, typically monovalent. Note that the base may be provided as its corresponding salt (preferably a carbonate) and the term ‘base’ as used herein should be construed accordingly where the context so permits. A preferred base is Tris (i.e. tris-hydroxymethyl methane). A preferred chelating agent is EDTA (i.e. ethylene diamine tetra-acetic acid). The free radical trap is less significant than the detergent, base and chelating agent. A suitable free radical trap is uric acid or a urate salt. It may be particularly useful in situations where the sample is not processed immediately after contacting with the assay device but is left for some time (e.g. to be archived) before any amplification reaction takes place. In other situations the free radical trap may normally be dispensed with.

In one embodiment, the lateral flow assay device in accordance with the first or second aspects of the invention, comprises an FTA matrix or similar, preferably in the sample receiving zone. FTA paper is available from Whatman International Limited (Maidstone, Kent, UK) and comprises a cellulose-based matrix coated with agents (such as those described above) which lyse cell and nuclear membranes, denature polypeptides and inactivate enzymes (such as nucleases) and which protect nucleic acid from UV-mediated or other environmental damage. PTA paper has been described in detail in the U.S. patents referred to in the preceding paragraph. A similar material, known as IsoCode®, is available from Scheicher & Schuell.

For the purposes of illustration, a suitable sample receiving/extraction zone matrix comprises a cellulose-based paper, such as filter paper, having a minimal loading (per square cm of paper) as follows: SDS or Triton X 100 1 mg; Tris 8 micromols (968.8 mg of free base); EDTA 0.5 micromols (146.1 mg free acid) and, optionally, uric acid 2 micromols (336.24 mg).

Nucleic acids present in the sample become temporarily entrapped within the matrix. Wash or carrier liquid (e.g. TE buffer) can be added to wash away the undesired contaminants. Care must be taken that the amount of fluid added is not so great as to saturate the lateral flow assay matrix and wick (normally present), otherwise subsequent capillary flow will not be possible.

The FTA paper or similar such material may be restricted to the sample receiving zone or may constitute all or a substantial part of the porous matrix of the lateral flow assay device. The amplification reaction may take place on the FTA paper—the reagents which inactivate enzymes etc. being washed off the matrix by the application of wash or carrier liquid. Alternatively, nucleic acid temporarily entrapped within the FTA matrix may be eluted (by application of for example, Tris-EDTA buffer or other aqueous EDTA-containing solution to the matrix) and thence transported into a downstream portion of the lateral flow device comprising a generally conventional nitrocellulose or similar porous matrix, within which the amplification reaction may be performed. Alternatively, in a device in accordance with the second aspect of the invention, the sample receiving zone may be an essentially inert conventional matrix (e.g. nylon or nitrocellulose) to which a pre-extracted nucleic acid-containing sample is applied.

Molecular techniques that involve the manipulation of nucleic acids may for certain applications use organosilicon oxide polymers to prevent loss of material by adsorption onto surfaces. Dichlorodimethylsilane, for example, is the active ingredient in silanising solutions used to coat microcentrifuge tubes, disposable pipette tips and the like to prevent loss of nucleic acids due to adhesion on the surfaces of these devices.

The inventors propose that specific surfaces utilised in lateral flow devices for use with nucleic acids may be treated with a silanising solution to prevent adhesion of nucleic acid based probes and/or analyte to the device matrix/ces. Matrices comprising, for example, glass fibre or plastics may be thus treated. Such treatment may also improve flow dynamics of the device.

It has been known since the early 1950's that DNA binds in a reversible manner to silica in the presence of chaotropic salts. The silicate is thought to interact with double stranded DNA by dehydrating the phosphodiester backbone by the chaotropic salts, which allows exposed phosphate residues to adsorb to the silica. Once adsorbed, the double stranded DNA remains in either a native or partially denatured (single stranded) state and cannot be eluted from the matrix by solvents that displace other biopolymers such as RNA and carbohydrate. However, immobilized DNA can be rehydrated and recovered by washing with aqueous buffers.

A further embodiment therefore may also include the use of silica (various grades) coated onto glass or plastic (as in thin-layer chromatography) as the matrix for nucleic acid based lateral flow tests. Various zones on the device may be created by the inclusion of e.g. sucrose gates to separate the various reaction zones. The thickness of the gates would determine the time reactants would spend in each zone thereby enabling specific reactions to progress to completion prior to emerging into distally placed zones. Nucleic acids may also be retained in specific zones by the use of chaotropic agents and released by the addition of aqueous buffers to enable migration into distally located zones.

The nucleic acid sequence of interest may comprise DNA, RNA, or mixtures thereof and may be a naturally occurring molecule or a synthetic molecule. Typically the sequence of interest may be derived from an infectious disease agent of man or animals, food spoilage organisms, or from animal (especially mammalian), human or plant sources. The assay devices find particular application as diagnostic tools to assist in diagnosis of infectious diseases or other pathological conditions (e.g. diagnosis of genetic disorders or conditions associated with particular genetic abnormalities) and in the detection of spoilage organisms in foods or detection of pathogens or markers of faecal contamination (e.g. E. coli) in water or other environmental samples. Thus the sample applied to the sample receiving zone of the assay device may be, or be derived from (as appropriate), any sample of interest. The sample may typically be a biological sample (e.g. blood, plasma, serum, urine, sweat or the like), or a sample of food or drink, or an environmental sample such as a water sample or a swab from a surface.

The lateral flow assay device of the invention is typically a low-cost item and disposed of after a single use. Generally the assay device comprises a permeable or porous matrix, or other liquid flow means, which at a proximal end is in liquid communication with the sample receiving zone, such that liquid applied to the sample receiving zone may flow along the device through the permeable or porous matrix by virtue of capillary action. It is conventional to provide a highly absorbent “sink” or wicking member at the distal end of the porous matrix, to enhance the capillary flow. Analytes and/or reagents suspended or dissolved in the liquid may be transported along the device by the flow of liquid.

Typically the porous matrix and wicking member are substantially enclosed within an impervious casing, often comprising a synthetic plastics material, to facilitate handling of the device and to protect the matrix against contamination.

Again, it is conventional to provide lateral flow assay devices with at least one test reagent which is releasably bound in and/or on the porous matrix, typically in dessicated or lyophilised form, such that contacting the assay device with a liquid will release the test reagent which may then be transported by the capillary flow of the liquid along the porous matrix. It is also conventional to provide at least one test reagent, typically a capture reagent, in the detection zone of the device, which reagent is immobilised in and/or on the porous matrix, such the flow of liquid along the matrix will not release the reagent in question. This facilitates concentration and detection of an analyte in the detection zone.

The assay devices of the present invention will normally possess these conventional features. The general principle of operation of the devices of the invention is that a nucleic acid sequence of interest present in a sample applied to the sample receiving zone will be transported, by capillary flow along the porous matrix to the nucleic acid amplification zone where, typically dependent on the presence of the sequence of interest, a nucleic acid amplification reaction will take place. The amplified product/s of that reaction (known as the amplicon/s) will typically become labelled in an amplicon-specific manner, and will continue to pass along the porous matrix to the detection zone, where the amplicon/s will be captured by an immobilised capture molecule.

Those skilled in the art will appreciate that it is necessary for liquid to be present in order to perform the assay. The sample may itself be in liquid form. Alternatively it may be necessary to add a carrier liquid to the sample either prior to contacting the sample with the assay device, or in situ on the sample receiving zone. The carrier liquid will normally be aqueous and may include, for example, distilled or deionised water, or an aqueous buffer solution, such as TE buffer. The carrier liquid may be added all in one go, or be added in discrete aliquots (this latter option may usefully be employed to help control the flow of the analyte and/or reagents along the assay device). Conveniently the carrier liquid may comprise one or more of the reagents required to perform the assay (e.g. RNA or DNA polymerases; rNTPs or dNTPs; probes; labels etc). In addition, or alternatively, as explained above, carrier liquid may be applied to the device to wash away contaminants from the sample receiving zone and/or to elute away from the sample receiving zone agents useful for performing the extraction step but which may inhibit the subsequent nucleic acid amplification reaction. The amount of carrier liquid applied to the device will typically be in the range 50 μl-2 ml, preferably in the range 100 μl-1 ml.

Generally, the arrangement will be such that liquid (together with associated analytes and/or resuspended reagents) will flow from one zone to another, allowing various steps of the assay to be performed sequentially. This flow may be essentially continuous at a substantially constant speed. However it may be preferred to cause a discontinuous flow, with different flow rates at different points along the porous matrix, e.g. to allow certain reaction products to accumulate before they proceed to the next zone of the device. Variation of the flow rate may be achieved by any of a number of suitable means, including but not limited to, a physical switch, a dissolvable barrier (e.g. sucrose), restriction of capillary flow (e.g. by altering the porosity/permeability of the matrix) and the like. Examples of fluid control systems used in immunoassay lateral flow assays, and which may be employed in the present invention, include the use of chemical gates (U.S. Pat. No. 6,271,040), centrifugal force (U.S. Pat. No. 4,989,832), capillary restrictions (U.S. Pat. No. 6,271,040), separate fluid channels of differing pathlengths for reagents (U.S. Pat. No. 4,960,691; U.S. Pat. No. 5,198,193), or physical means (e.g. the WheatRite test from C-Qentec). The porous matrix may be provided as a single continuous strip or may be formed from two or more portions which are held, or locatable, in liquid communication so as to provide a liquid flow path from one portion to an adjacent portion.

In one embodiment the lateral flow assay device comprises means to alter the relative positions of two or more portions of the porous matrix, so as to affect the rate of flow of liquid from one portion to another. This may comprise, for example, a plunger or push-button which can be actuated to bring previously separated portions of the matrix into liquid flow communication with one another. Alternatively, the device may comprise a foldable portion such that two components are not in liquid flow communication in a first conformation, but that folding the foldable portion of the device (e.g. along a scored line or laterally flattened fold line) will bring the previously separate components into liquid flow communication.

In addition, or as an alternative, to affecting the flow rate between different portions of lateral flow device, means may be provided to affect the flow path of a liquid within the device. For example, the flow path could comprise one or more branch points, at which a liquid could flow in one of two or more different directions, and the assay device could comprise means for influencing the flow path adopted by the liquid. In one embodiment, two or more porous or bibulous members are provided downstream of a branch point, each downstream porous or bibulous member presenting a possible flow path. If desired one or more of the downstream porous or bibulous members can be provided such that in an initial state, they are not in liquid flow communication with the branch point, but can be brought into such liquid flow communication subsequently e.g. by removal of liquid impermeable barrier (such as a thin film of synthetic plastics material) or by a user actuating a switch mechanism (e.g. applying pressure against a biassing means so as to close a gap or space between the branch point and the downstream member). Alternatively a downstream member may initially be in liquid flow communication but is forced out of such communication by physical separation (e.g. moving a switch or biassing means) or by inserting an impermeable barrier. In this way, a liquid can be diverted between various liquid flow paths, as desired.

The porous matrix may comprise, for example, cellulose and/or cellulose derivatives (especially nitrocellulose), although any suitable porous material (e.g. glass fibre, nylon, polysulfone) may be used. Preferably the porous matrix is provided with a backing material (typically a piece of plastics sheet material, such as Mylar®) to provide increased strength and rigidity. Typically the porous matrix may be treated with conventional agents to prevent non-specific binding/absorption of analyte or reagents. Suitable blockers of non-specific binding include polyvinyl alcohol (PVA) and polyvinyl pyrrolidone (PVP).

In some embodiments the lateral flow assay device will comprise at least one reagent which is required for the nucleic acid amplification reaction, which reagent is provided releasably bound to the porous matrix in, or upstream of, the amplification zone. In some embodiments at least one reagent, required for the nucleic acid amplification reaction, is provided suspended or dissolved in a carrier liquid which is applied (typically at the sample receiving zone) to the lateral flow assay device. Moreover, it is also possible that at least one reagent required for the amplification reaction may be immobilised on or in the porous matrix (i.e. such that flow of a liquid along the matrix will not release the reagent). For example, oligonucleotide or polynucleotide probes, primers and the like may be immobilised to an amino-activated matrix by phenyldiisothiocyanate (PITC) or disuccinimidyl suberate.

It will be noted that the embodiments described immediately above are not mutually exclusive and may be combined in any combination e.g. wherein one or more reagents requires for the nucleic acid amplification reaction may be releasably bound to the matrix, one or more may be immobilised on or in the matrix, whilst one or more other reagents may be present in a liquid applied to the lateral flow assay device. Typically the amplification reaction will require: the target sequence of interest; at least one nucleic acid probe which comprises a portion complementary to the target sequence; at least one nucleic acid polymerase; and nucleotide triphosphates which may be utilised by the nucleic acid polymerase to synthesise a polynucleotide or oligonucleotide.

The nucleic acid amplification zone is that part of the assay device in which all the essential components of the amplification reaction are brought together so that, in suitable conditions, the amplification reaction occurs. Thus, the amplification zone may or may not be a clearly discernible or discrete portion of the lateral flow assay device. In particular, the amplification zone may be co-extensive with, or form part of, the sample receiving zone.

Further the amplification reaction may be one which results in amplification (i.e. synthesis of multiple copies) of the target sequence or one which results in amplification of a signal sequence, generation of the signal sequence being ultimately dependent on the presence of the target sequence of interest in the sample. Examples of target sequence amplification techniques which may be employed include PCR, NASBA (U.S. Pat. No. 5,130,238) and TMA (U.S. Pat. No. 5,399,491). Examples of signal sequence amplification techniques which may be employed include SMART (WO93/06240) SPAR (WO99/37805) and Invader/Cleavase (U.S. Pat. No. 5,846,717).

In a device in accordance with the second aspect of the invention it is an essential feature that the amplification reaction is an isothermal reaction (i.e. one performed at a substantially constant temperature, without thermal cycling). In a device in accordance with the first aspect of the invention it is a preferred feature that the amplification reaction is an isothermal reaction. The isothermal amplification reaction may take place at room temperature (e.g. 20° C.) or may take place at some other temperature. In order to increase the speed of the reaction and/or increase the stringency of hybridisation it may be preferred to perform the reaction at an elevated temperature (e.g. at a temperature in the range 30-50° C.). Since thermal cycling is not required a simple ‘hot block’, oven, water bath or other incubator may be used to heat the assay device and hold it at the desired temperature for the requisite period of time.

A preferred isothermal amplification technique is a signal amplification method. In particular, preferred amplification reactions comprise SMART (as disclosed in WO 93/06240) and/or SPAR (as disclosed in WO 99/37805). Both these techniques require the use of at least one nucleic acid probe which comprises a sequence which is complementary to the sequence of the target of interest.

In the case of SMART, two such probes are employed, each being complementary to a different, but adjacent portion of the target nucleic acid, such that in the presence of the target the two probes (one a “template” probe, the other an “extension” probe) become hybridised adjacent to each other on the target, in a complex known as a “three way junction”. The hybridisation of the two probes in close proximity allows the further hybridisation of respective ‘arm’ portions of the probes to each other. One of these arms (the arm of the “template” probe) is longer than the other (the shorter arm being that of the “extension” probe). This allows the shorter of the two arms to be extended, using the larger arm as a template, by a DNA-dependent DNA polymerase in the presence of dNTPs. Extension of the arm creates a double stranded portion of nucleic acid which comprises an RNA polymerase promoter sequence (e.g. one recognized by T7, T3 or SP6 RNA polymerases).

Thus, in the presence of a suitable RNA polymerase and rNTPs, multiple RNA copies of one of the probes are formed. This results in “signal” amplification, and the multiple RNA copies may themselves be further amplified, if desired, by any one of a number of amplification processes known to those skilled in the art (e.g. as disclosed in WO 01/09376). The RNA copies, or amplified copies thereof, may then typically be captured and detected in the detection zone.

Accordingly, both dNTPs, rNTPs, DNA polymerase, RNA polymerase and suitable buffers may be required, as well as template and extension probes. Conveniently, the majority of these reagents will be provided in a carrier liquid applied to the sample receiving zone of the assay device and/or releasably bound to the porous matrix of the device. In one embodiment one of the probes (preferably the template probe) is immobilised in or on the porous matrix and the other reagents are present in a carrier fluid applied to the sample receiving zone and/or releasably bound to the porous matrix.

Labelling

Conveniently the amplicon/s (i.e. the amplified end product/s of the amplification reaction) becomes associated with a readily detectable label upstream of the detection zone. The label may be any suitable substance that is readily detectable e.g. a radio label or an enzyme label. It is however greatly preferred that the label is a direct visible label (i.e. one which is apparent to an observer without any prior processing) such as particulate coloured “latex” (in actuality, these “latex” particles are polystyrene) or colloidal gold particles.

It is desirable that the labelling is amplicon-specific. One of the simplest ways of achieving this is to ensure that the amplicon has a sequence which is essentially unique amongst the nucleic acids entering a labelling zone and to provide a labelling reagent which comprises a base sequence complementary to that of the amplicon, such that the labelling reagent hybridises to the amplicon in a sequence-specific manner.

Desirably the labelling reagent is provided releasably bound to the porous matrix, upstream of the detection zone, such that as amplicon migrates along the assay device it becomes associated with the labelling reagent which is released by the capillary flow of liquid, the complex of amplicon and labelling reagent then migrating to the detection zone.

The labelling moiety will conveniently comprise, in addition to the label, a moiety which is a member of a specific binding pair (“sbp”). Such sbps are well known to those skilled in the art and include antigens/antibodies, complementary strands of nucleic acid, ligands/receptors and the like. A preferred sbp for present purposes is biotin/strepavidin.

Detection

The labelled amplicon is detected in the detection zone. This is conveniently achieved by immobilising on the porous matrix a capture molecule which is specific for the labelled amplicon complex (more particularly, specific for the amplicon). The amplicon-specific capture molecule may be any molecule which can bind in a specific manner to the amplicon and which may be immobilised on the porous matrix. Conveniently the amplicon-specific capture molecule may comprise a nucleic acid sequence complementary to that of the amplicon or may comprise a nucleic acid binding protein (e.g. a “zinc finger” polypeptide) or a sequence-specific anti-DNA or anti-RNA antibody (or effective binding portion thereof, such as an Fab, Fv, scFv etc.).

The amplicon-specific capture molecule will conveniently be immobilised in a line or band across the porous matrix or other recognisable location, preferably substantially tranversely arranged relative to the direction of liquid flow along the assay device. Accordingly, labelled amplicon will be captured and concentrated, forming a visible line in the detection zone. It is perfectly possible, however, to deposit the capture molecule in other configurations, so as to form, for example, a spot or other shape. In addition, it is possible to arrange the device so as to deposit a capture molecule in two or more locations. If desired, two or more different capture molecules may be deposited at respective locations, each capture molecule being specific for a respective amplicon, such that a single device can be used to test for the presence and/or amount of a respective number of different sequences of interest. Naturally, the amplification reagents to amplify the different targets (or sequences derived therefrom) will need to be provided also.

It is also preferred that the assay device comprises a control zone, advantageously downstream of the detection zone. The control zone typically comprises an immobilised capture reagent which binds specifically to a reagent which participates in the amplification and/or detection reactions or which might be generated by a control nucleic acid amplification reaction. One convenient arrangement is for the control zone to comprise a line or band of immobilised reagent which exhibits specific binding for the labelling reagent (e.g. the labelling reagent may be a biotinylated olignucleotide and the immobilised control zone capture molecule comprises streptavidin).

In an alternative embodiment the capture zone comprises an immobilised array of capture molecules which capture excess labelled amplicon.

For the avoidance of doubt it is explicitly stated that any feature of the invention described herein as “preferred”, “advantageous”, “desirable”, “convenient” or the like may be incorporated in an embodiment of the invention either in combination with any other feature so described or in isolation.

The invention will now be further described by way of illustrative example and with reference to the accompanying drawings, in which:

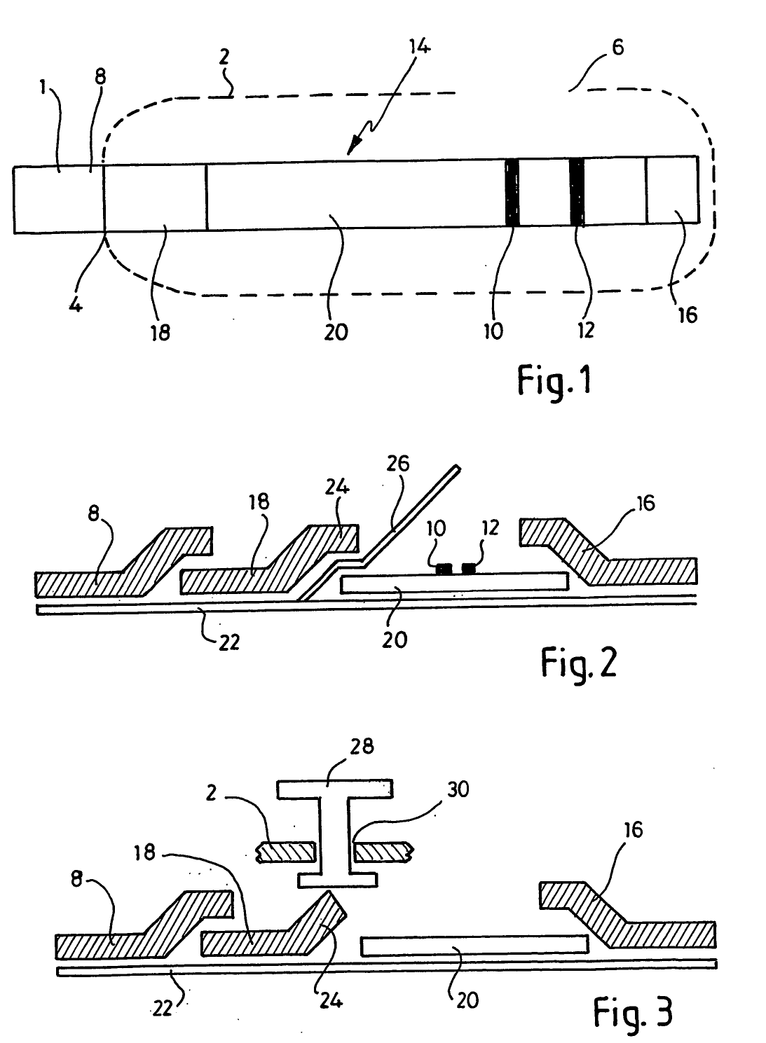

FIGS. 1, 2 and 3 are schematic representations of various embodiments of an assay device in accordance with the first and/or second aspects of the invention;

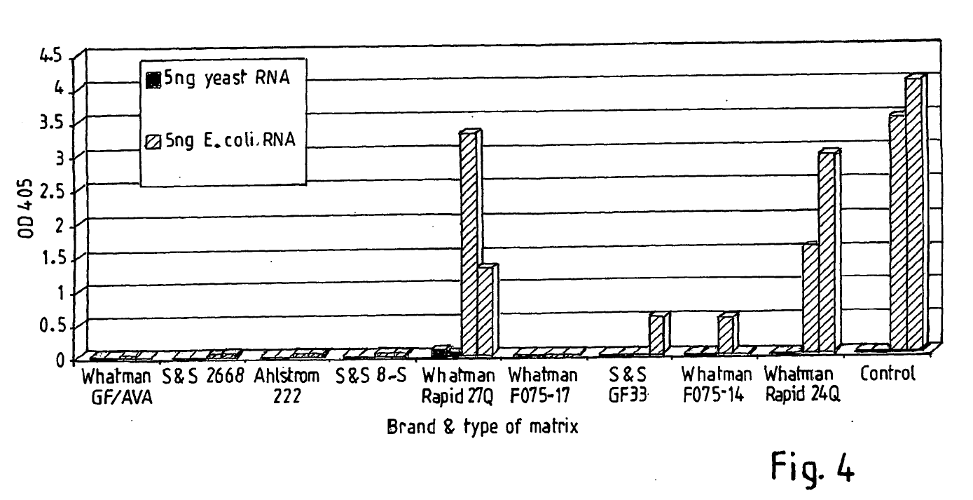

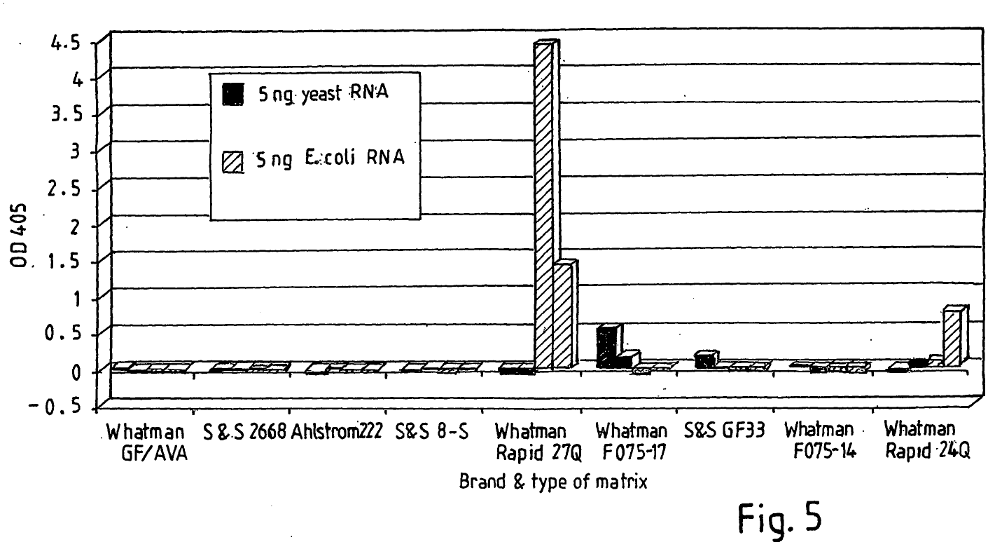

FIGS. 4 and 5 are bar charts showing the amount of signal (as measured by ELOSA, enzyme-linked oligosorbent assay) obtained using different porous matrices;

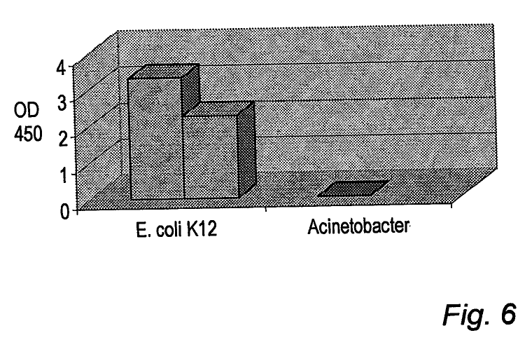

FIG. 6 is a bar chart showing the amount of signal (as measured by ELOSA) obtained following a target-specific amplification reaction.

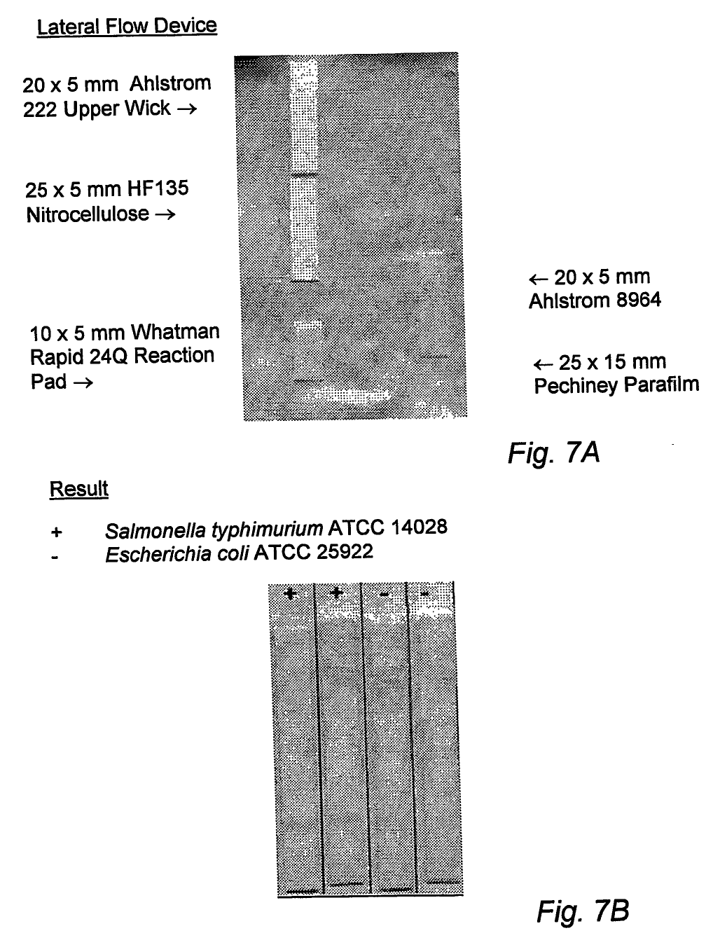

FIGS. 7A, 8A and 9A show the components used to make a simple lateral flow assay device and FIGS. 7B, 8B and 9B are pictures of the assembled components showing results obtained.

### EXAMPLES

### Example 1

This example demonstrates that the method essentially outlined in WO 99/37806 (SMART) can take place on samples of solid matrices as would be used in a lateral flow device. E. coli 23S rRNA was used as target for this example.

Preparation of Oligonucleotides

All oligonucleotide probes were synthesised by phosphoramidite chemistry using an Applied Biosystems 380A synthesiser according to the manufacturer's instructions. Octanediol incorporation was accomplished by reaction of the growing chain with Octanediol-phosphoramidite (Oswel). Biotinylation of oligonucleotide probes was achieved by incorporation of a biotin phosphoramidite. Oligonucleotides functionalised with Alkaline Phosphatase were prepared using the manufacturer's proprietary method (Oswel). All oligonucleotides were HPLC purified using standard techniques.

Preparation of RNA

RNA for the positive reactions was prepared from the strain E. coli K12 with the Qiagen RNeasy total RNA preparation kit, using manufacturer's instructions. RNA was quantified using Ribogreen (Molecular Probes) fluorescent stain, according to manufacturer's instructions. Commercial yeast RNA was used for the negative controls (Roche).

SMART Reaction on Solid Matrices

Discs of each matrix (6 mm in diameter) were prepared using a standard paper hole punch. In order to ensure the matrix absorbed the entire SMART reaction volume, a 3× disc-layer sandwich of each matrix type was used per SMART reaction. This sandwich was horizontally positioned in a 0.2 mL reaction tube, at the level where the tube begins to taper. The different matrices tested were: Whatman GF/AVA; S&S 2668; Ahlstrom 222; S&S 8-S; Whatman Rapid 27Q; Whatman F075-17; S&S GF33; Whatman F075-14; and Whatman Rapid 24Q.

A start mix was made comprising (per reaction) mixes ‘A’ & ‘C’ (5 μl of each mix). A 100 μl master mix of mix A comprised of 1.0 nM probe 1; 0.2 nM probe 2; 20 nM probe 3; 20 nM probe 4; 4.0 nM probe 5; 180 nM probe 7; 16 mM of each rNTP (A, G, U, C); 0.22 mM of each dNTP (A, G, T, C); 5% sucrose; 1% ficoll; 1% PVP; 5 μl TE (10 mM Tris & 1.0 mM EDTA, pH 8.0); made up to volume with molecular grade water. A 100 μl master mix of mix C comprised of 160 mM Tris (pH 7.8); 24 mM MgCl₂; 8 mM spermidine; 40 mM DTT; 600 mM NaCl, made up to volume with molecular grade water. The combined A&C start mix per reaction (total 10 μl) was mixed with 5 μl of TE containing either 5 ng of E. coli K12 RNA (positive), or 5 ng of yeast RNA (negative). The resulting 15 μl mixture was added to the matrix sandwich in the 0.2 mL reaction tube. For control reactions, the 15 μl mixture was added directly to 0.2 mL reaction tubes. The reaction tubes were placed in a thermal cycler, and heated to 95° C. for 5 min, followed by cooling to 41° C. at 0.1° C. sec⁻¹. The reactions were incubated at 41° C. for 60 min. During the 60 min stage, a master mix of enzyme (mix D) and enzyme diluent (mix E) was prepared. Per reaction, this consisted of, for mix D: 2 μl (=400 units) T7 RNA polymerase (Ambion); 1 μl (=8 units) Bst DNA polymerase (New England Biolabs), and for mix E: 66.67 mM Tris (pH 7.8); 10 mM MgCl₂; 3.33 mM spermidine; 16.67 mM DTT; 5.56% sucrose; 1.11% ficoll; 1.11% PVP, made up to volume (27 μl) with molecular grade water. 30 μl of the enzyme/enzyme diluent mix (D/E) was added to the matrix sandwich in the reaction tubes (or directly into reaction tubes in the case of the controls), and these were incubated at 41° C. for 90 min.

Capture and Detection of Synthesised RNA

For detection of the RNA signal, two samples were taken for each reaction. The first consisted of liquid that had collected at the bottom of the reaction tubes. The second consisted of the matrix sandwich itself, added to the end detection reaction. Capture and detection of the resulting RNA signal was achieved by ELOSA as follows:

Probe 6 (3-6 pmol) was added to 55 μl of solution H (hybridisation buffer), which consisted of 20 mM EDTA (pH8.0); 1.0M NaCl; 50 mM Tris; 0.1% bovine serum albumin (Sigma), adjusted to pH 8.0 with HCl and made up to volume with molecular grade water. The liquid samples from the reactions were added to the wells of a streptavidin-coated microtitre plate (Thermo Life Sciences), followed by the 55 μl of solution H+probe 6.

The matrix samples were added to microtitre plate wells already containing 55 μl of solution H+probe 6. The wells were sealed with an adhesive disposable plastic plate-sealer, and incubated at room temperature with shaking (200 rpm) for 30 min. The plate sealer was removed and discarded, and the contents of the wells were also discarded. The wells were washed 2× with 200 μl of solution W (wash solution), consisting of 50 mM Tris; 138 mM NaCl; 2.68M KCl; 21.34 mM MgCl₂; 0.1% Tween 20, adjusted to pH 8.0 with HCl and made up to volume with sterile distilled water. For the final colour reaction, 100 μl of solution S (substrate buffer) containing 5 mg/mL of pNitrophenyl phosphate (pNpp) was added to each well. Solution S consisted of 1M diethanolamine; 21.32 mM MgCl₂; 15.38 mM sodium azide, adjusted to pH 9.8 with HCl and made up to volume with sterile distilled water, with one pNpp tablet (Sigma cat. no. N2765) dissolved in 4 mL of solution S. The wells were sealed with a fresh plate sealer, and the plate was incubated at 37° C. for 15 min. The colour reaction was stopped by adding 100 μl of solution T (stop solution), which consisted of a 30.5 mL 1M Na₂HPO₄ added to 19.5 mL 1M NaH₂PO₄ (sodium phosphate buffer pH 7.0). Optical density of the colour reaction was read at OD₄₀₅ nm.

List of Oligonucleotides

Probe 1 (extension)
5′ GCATTTAGCTACCGGGCAGTGCCATTTTCGAAAT 3′
Probe 2 (template)
5′ TCGTCTTCCGGTCTCTCCTCTCAAGCCTCAGCGCTCTCTCTCCCTATAGTGAGT
CGTATTAATTTCGAA-(octanediol)-GGCATGACAACCCGAACACCAGTGAT 3′ (3′ phosphorylation)
Probe 3 (facilitator 1)
5′ GCG TCC ACT CCG GTC CTC TCG 3′ (3′ PCR-block)
Probe 4 (facilitator 2)
5′ GCTTAGATGCTTTCAGCACTTATCTCTTCC′3 (3′ PCR-block)
Probe 5 (linear)
5′ TGCCTGCTTGTCTGCGTTCTGGATATCACCCGAGTTCTCGCTTCCTATAGTGA
GTCGTATTAATTTCTCGTCTTCC-(octanediol)-
GGTCTCTCCTCTCAAGCCTCAGCGCTCTCTCTCCC 3′ (3′ phosphorylation)
Probe 6
5′ GGATATCACCCG 3′ (5′ Alkaline Phosphatase labelled)
Probe 7
5′ TCTGCTGCCTGCTTGTCTGCGTTCT 3′ (5′ biotin labelled)

The results are shown in FIGS. 4 and 5.

Conclusions

Positive SMART signals, indicating detection of E. coli 23S rRNA, were obtained from solid phase reactions on Whatman Rapid 27Q and Whatman Rapid 24Q matrices (FIG. 4). Low positive signals were obtained on one of the two duplicate solid phase reactions using S&S GF33 and Whatman F075-14 matrices. Liquid collected from the bottom of reaction tubes showed positive signal from tubes containing Whatman 27Q matrix (FIG. 5). One duplicate reaction of the liquid collected from the bottom of reaction tubes containing Whatman 24Q matrix gave a low positive signal.

### Example 2

This example demonstrates detection of target nucleic acid following lysis of bacterial cells and putative immobilization of the nucleic acid by Whatman FTA paper using the isothermal nucleic acid amplification method essentially described in WO 99/37806 (SMART). It also demonstrates that addition of SMART reagents to Whatman FTA paper (FTA® Classic Card), following said lysis and immobilization, results in a target-specific SMART reaction.

Preparation of Oligonucleotides

All oligonucleotide probes were synthesised and purified as described in Example 1. Additionally, oligonucleotides functionalised with Horse Radish Peroxidase were prepared using the manufacturer's proprietary method (Oswel).

Preparation of RNA

Cells for the positive reactions were prepared by incubation of E. coli K12 in Nutrient Broth (Oxoid) for 16 hours at 37° C. Cells for the negative reaction were prepared by incubation of Acinetobacter spp. in Nutrient Broth (Oxoid) for 16 hours at 37° C.

Cell Iysis

1. Using a pipette, 5 μl sample of broth was added to FTA paper, and allowed to air dry for one hour.
2. A 2 mm disc of the paper containing the sample was made using a Harris Micro Punch tool. The punch was transferred to a reaction tube (0.2 ml).
3. 200 μl of FTA Purification Reagent (Whatman) was added and mixed by pipetting.
4. The tube was capped and incubated for 5 min at room temperature.
5. The Purification Reagent was removed with a pipette.
6. Steps 3-5 were repeated a further two times giving a total of three washes
7. After the final wash and removal of Purification Reagent, 200 μl TE was added to the tube and mixed by pipetting.
8. The tube was capped and incubated for 5 min at room temperature
9. The TE was removed with a pipette.
10. Steps 7-9 were repeated.
11. The punch was allowed to completely air dry for 60-90 min at room temperature.
   SMART Reaction

A start mix was made comprising (per reaction) mixes ‘A’ & ‘C’ (5 μl of each mix). The composition of master mixes A and C was as described for Example 1, but using the probe set listed below. 5 μl of molecular grade water was added to the punch. The combined A&C start mix per reaction (total 10 μl) was added to the punch in the 0.2 mL reaction tube, and subjected to thermal transitions as described in Example 1. The reactions were incubated at 41° C. for 60 min. following which 30 μl of the enzyme/enzyme diluent mix (D/E), as detailed in Example 1, was added to the tube containing the punch in the reaction tubes, and these were incubated at 41° C. for 90 min.

Capture and Detection of Synthesised RNA

Capture and detection of the resulting RNA signal was achieved by ELOSA as follows:

Probe 6 (0.1 pmol) was added to 55 μl of solution H (hybridisation buffer), which consisted of 20 mM EDTA (pH8.0); 1.0M NaCl; 50 mM Tris; 0.1% bovine serum albumin (Sigma), adjusted to pH 8.0 with HCl and made up to volume with molecular grade water. The liquid samples from the reactions were added to the wells of a streptavidin-coated microtitre plate (Thermo Life Sciences), followed by the 55 μl of solution H+probe 6. The matrix punch samples were added to microtitre plate wells already containing 55 μl of solution H+probe 6. The wells were sealed with an adhesive disposable plastic plate-sealer, and incubated at room temperature with shaking (200 rpm) for 30 min. During incubation, the liquid colour substrate 3,3′,5,5′-tetramethylbenzidine (TMB: Sigma) was taken from storage at 4° C., to allow equilibration to room temperature. The plate sealer was removed & discarded, and the contents of the wells were also discarded. The wells were washed 2× with 200 μl of solution W (wash solution), consisting of 137 mM NaCl; 2.68M KCL; 10 mM Na₂HPO₄; 2.0 mM KH₂PO₄, adjusted to pH 7.4 with HCl, 0.05% Tween 20, and made up to volume with sterile distilled water. For the final colour reaction, 100 μl of TMB was added to each well. The colour reaction was incubated room temperature 5 min. The colour reaction was stopped by adding 100 μl 1N HCl. Optical density of the colour reaction was read at OD₄₅₀ nm.

List of Oligonucleotides for Example 2

Probe 1 (extension)
5′ GCATTTAGCTACCGGGCAGTGCCATTTTCGAAAT 3′
Probe 2 (template)
5′ TCGTCTTCCGGTCTCTCCTCTCAAGCCTCAGCGCTCTCTCTCCCTATAGTGAGT
CGTATTAATTTCGAA-(octanediol)-GGCATGACAACCCGAACACCAGTGAT 3′ (3′ phosphorylation)
Probe 3 (facilitator 1)
5′ GCGTCCACTCCGGTCCTCTCG 3′ (3′ PCR-block)
Probe 4 (facilitator 2)
5′ GCTTAGATGCTTTCAGCACTTATCTCTTCC 3′ (3′ PCR-block)
Probe 5 (linear)
5′ TGCCTGCTTGTCTGCGTTCTGGATATCACCCGAGTTCTCGCTTCCTATAGTGA
GTCGTATTAATTTCTCGTCTTCC-(octanediol)-
GGTCTCTCCTCTCAAGCCTCAGCGCTCTCTCTCCC 3′ (3′ phosphorylation)
Probe 6
5′ GGATATCACCCG 3′ (3′ Horse Radish Peroxidase - labelled)
Probe 7
5′ TCTGCTGCCTGCTTGTCTGCGTTCT 3′ (5′ biotin labelled)

The results are shown in FIG. 6.

Conclusions

Lysis of E. coli K12 cells on Whatman PTA paper, followed by immobilisation by air-drying, resulted in a positive reaction by a SMART reaction containing 3WJ probes specific to 23S rRNA. Acinetobacter generated no signal.

### Example 3

This example demonstrates that a synthetic DNA homologue of the signal (RNA1) from a SMART reaction (WO 99/37806) could be detected on a lateral flow device (dipstick).

Preparation of Oligonucleotides

All oligonucleotides probes were synthesised and purified as described in Example 1. Additionally, oligonucleotides labelled with dinitrophenol (DNP) were prepared using the manufacturer's proprietary method (Oswel).

Detection of a Synthetic Target Using a Lateral Flow Device (Dipstick)

Nitrocellulose dipsticks (Schleicher & Schuell) of 20 mm length, 5 mm width and a pore size of 5-12 μm, were impregnated with a line (0.5 mm width) of anti-biotin antibody 10 mm from the base of the stick.

Hybridisation Step

In a 0.2 mL reaction tube was mixed 2 μl 5× transcription buffer (Promega, 200 mM Tris pH 7.9, 30 mM MgCl₂, 10 mM Spermidine and 50 mM NaCl) and 1 μl of a 1 μM solution in water of the biotinylated capture probe 4 and the DNP labelled detection probe 6 (see “list of Oligonucleotides”).

Following the addition of 1 μl of molecular grade water, a dilution series of synthetic target probe 3 (5 μl of a set of 2 fold dilutions starting from 40 nM and ending at 6.5 nM with a 0 nM negative control.). The probes were allowed to hybridise at room temperature for 15 minutes.

Development Step

A suspension of blue coloured latex beads functionalised with anti-DNP 5 μl (100 μg/ml, 0.5% solids-batch number NK220500) was mixed with 50 μl latex bead diluent (50 mM Tris, 0.1% Tween pH 7.9). The hybridisation mix was added to the diluted latex bead suspension, and then transferred to the well of a microtitre plate. A test strip was placed into the well and the solution front was allowed to migrate to the top of the strip.

Results

The development, within 2 minutes, of a thin blue line located 10 mm from the base of the strip confirmed a positive detection of synthetic target. No blue line was observed for the zero target control reaction.

Conclusion

By use of a set of DNP and biotin labelled specific probes for capture and labelling with blue latex beads, 25 fmols of synthetic target was detected.

### Example 4

This example demonstrates that the RNA signal (RNA1) generated from a SMART reaction (WO 99/37806) could be detected on a lateral flow device (dipstick).

Preparation of Oligonucleotides

All oligonucleotides were synthesised and purified as described in Example 1. Additionally, Hex incorporation was accomplished by reaction of the growing chain with 18-dimethoxytrityl hexaethylene glycol, 1-((2-cyanoethyl)-(N,N-diisopropyl))-phosphoramidite.

Detection of Transcribed RNA Target Using a Lateral Flow Device

RNA 1 amplicon was prepared from a SMART reaction and quantified using ELOSA (see Examples 1 and 2), by comparing signal from the reaction to a standard curve of synthetic target DNA.

SMART Reaction

Two start mixes, A and B, were prepared. Start mix A comprised (per reaction) 0.49 nM probe 10; 0.1 nM probe 11; 77.7 mM Tris (pH 7.8), 11.65 mM MgCl₂, 3.88 mM Spermidine, 19.4 mM DTT, and 290 mM NaCl, made up to 10.3 μl with molecular grade water. (See “list of Oligonucleotides”).

To start mix A, for positive reactions, 0.1 nM probe 12 (synthetic target) in 5 μl of molecular grade water was added. The control was 5 μl of molecular grade water. The components were mixed followed by a short centrifugation step to ensure all liquid was at the bottom of the reaction tube.

Tubes containing start mix A plus positive or negative targets were placed in a thermal cycler and heated to 95° C. for 5 min, followed by cooling to 41° C. at 0.1° C. sec⁻¹. The reactions were incubated at 41° C. for 60 minutes. During this time, start mix B was prepared. Start mix B comprised per reaction: 21.28 μM of each dNTP (A, G, T, C), 8.51 mM of each rNTP (A, G, U, C), 1.2 μl (240 units) of T7 RNA polymerase (Ambion) and 0.5 μl (4 units) Bst DNA polymerase (New England Biolabs) (total volume=4.7 μl). 4.7 μl of start mix B was added per reaction to the reaction tubes containing start mix A and the positive or negative targets.

The reactions were mixed by pipetting then incubated for 3 hours at 41° C.

The dipstick protocol and reagents used were as described in Example 3 except SMART RNA 1 amplicon (prepared as described above) was used as target instead of a synthetic target.

Results

The development, within 2 minutes, of a thin blue line located 10 mm from the base of the stick confirmed a positive detection of SMART RNA 1 amplicon for the positive target. No line was observed for the negative target reaction.

Conclusion

By use of a set of DNP and biotin labelled specific probes for capture and labelling with the blue latex beads, 50 fmols of SMART RNA 1 amplicon was detected.

### Example 5

This example demonstrates that a lateral flow device could detect RNA signal amplicon (RNA 2) from a SMART reaction (WO 99/37806) designed to detect E. coli K12.

Preparation of Oligonucleotides

All oligonucleotides probes were synthesised and purified as described in the preceding examples.

Detection of Transcribed RNA from a SMART Reaction Using a Lateral Flow Device

50 ng E. coli K12 RNA was detected by a SMART reaction to yield an RNA 2 signal amplicon.

SMART Three Way Junction (TWJ) Reaction

Two start mixes, A and B, were prepared. Start mix A comprised (per reaction) 0.49 nM probe 1; 0.1 nM probe 2; 77.7 mM Tris (pH 7.8), 11.65 mM MgCl₂, 3.88 mM Spermidine, 19.4 mM DTT, and 290 mM NaCl, made up to 10.3 μl with molecular grade water. To start mix A, for positive reactions, 50 ng of E. coli K12 genomic DNA in 5 μl of molecular grade water was added. The negative control was 50 ng of Micrococcus genomic DNA in 5 μl molecular grade water. The components were mixed followed by a short centrifugation step to ensure all liquid was at the bottom of the reaction tube.

Tubes containing start mix A plus positive or negative targets were placed in a thermal cycler (MJ Research) and heated to 95° C. for 5 min, followed by cooling to 41° C. at 0.1° C. sec⁻¹. The reactions were incubated at 41° C. for 60 minutes. During this time, start mix B was prepared. Start mix B comprised per reaction: 21.28 μM of each dNTP (A, G, T, C), 8.51 mM of each rNTP (A, G, U, C), 1.2 μl (240 units) of T7 RNA polymerase (Ambion) and 0.5 μl (4 units) Bst DNA polymerase (New England Biolabs) (total volume=4.7 μl). 4.7 μl of start mix B was added per reaction to the reaction tubes containing start mix A and the positive or negative targets.

The reactions were mixed by pipetting then incubated for 3 hours at 41° C.

SMART Linear Reaction

During the SMART TWJ reaction incubation, mix C was prepared. This comprised (per reaction) 5.88 μM of each dNTP (A, G, T, C); 2.35 mM of each rNTP (A, G, U, C); 0.77 μl (154 units) of T7 RNA polymerase (Ambion) and 0.5 μl (4 units) of Bst DNA polymerase (New England Biolabs); 105.9 mM Tris (pH 7.8); 15.9 mM MgCl₂; 5.3 mM Spermidine and 26.5 mM DTT, made up to 17 μl with molecular grade water.

Following completion of the TWJ reaction, the block temperature was reduced to 37° C. and 8 μl of a 2.5 nM solution of the probe 5 was added directly into the tubes in the block with mixing.

Mix C (17 μl) was then added, mixed by pipetting, followed by a further incubation for 2 hours at 37° C.

Hybridisation Assay

In a fresh 200 μl reaction tube, 20 μl of the SMART linear reaction was mixed with 1 μl of a 1 μM solution in water of the biotinylated capture probe 8 and the DNP labelled detection probe 9. The probes were allowed to hybridise at room temperature for 15 minutes. A control experiment in which probe 3, a synthetic analogue of the RNA 1 transcribed in the SMART TWJ reaction, was also prepared.

Development Step

A suspension of blue coloured latex beads functionalised with anti-DNP, 5 μl (100 μg ml⁻¹, 0.5% solids-batch number NK220500) was mixed with 50 μl latex bead diluent (50 mM Tris, 0.1% Tween pH 7.9).

The hybridisation mix was added to the diluted latex bead suspension and then transferred to the well of a microtitre plate.

A test strip was placed into the well and the solution front was allowed to migrate to the top of the strip.

Results

The development within 2 minutes of a thin blue line located 10 mm from the base of the stick confirmed a positive detection of E. coli K12. No line was observed for the Micrococcus negative control reaction.

Conclusion

By use of a set of DNP and biotin labelled specific probes for capture and labelling with blue latex beads, SMART RNA 2 amplicon was detected as a thin blue line on the strip indicating a positive detection of E. coli K12.

The positive line observed on the strip was as intense as the 200 fmol signal obtained from probe 7.

List of Oligonucleotides for Examples 3, 4 and 5

The following oligonucleotides were employed in performing one or more of Examples 3, 4 and 5.

Probe 1 (extension)
5′ GCATTTAGCTACCGGGCAGTGCCATTTTCGAAAT 3′
Probe 2 (template)
5′ TCGTCTTCCGGTCTCTCCTCTCAAGCCTCAGCGCTCTCTCTCCCTATAGTGAGT
CGTATTAATTTCGAA-(octanediol)-GGCATGACAACCCGAACACCAGTGAT 3′ phosphorylated
Probe 3 (DNA homologue of SMART RNA 1 amplicon)
5′ GGGAGAGAGAGCGCTGAGGCTTGAGAGGAGAGACCGGAAGACGA3′
Probe 4 (5′ biotinylated capture probe for SMART RNA 1 amplicon)
5′ TCTGCTCGTCTTCCGGTCTCTCCTC 3′
Probe 5 (linear)
5′ TGCCTGCTTGTCTGCGTTCTGGATATCACCCGAGTTCTCGCTTCCTATAGTGA
GTCGTATTAATTTCTCGTCTTCC-(octanediol)-
GGTCTCTCCTCTCAAGCCTCAGCGCTCTCTCTCCC 3′ phosphorylated
Probe 6 (3′ DNP labelled detection probe for SMART RNA 1 amplicon)
5′ GCCTCAGCGCTCTCTCTCCC 3′ DNP
Probe 7 (DNA homologue of SMART RNA 2 amplicon)
5′ GGAAGCGAGAACTCGGGTGATATCCAGAACGCAGACAAGCAGGCA
Probe 8 (5′ biotin capture probe for SMART amplicon RNA 2)
5′ TCTGCTGCCTGCTTGTCTGCGTTCT 3′
Probe 9 (3′ DNP labelled detection probe for SMART amplicon RNA 2)
5′ TCACCCGAGTTCTCGCTTCC 3′
Probe 10 (extension probe for RNA 1 generation)
5′ GCCTGGCACCATTAAAGAAAATATCATCTTTTTCGAAAT 3′
Probe 11 (template probe for RNA 1 generation)
5′ TGCCTGCTTGTCTGCGTTCTGGATATCACCCGAGTTCTCGCTTCCTATAGTGA
GTCGTATTAATTTCTCGTCTTCC-(hexaethyleneglycol)-
GGTCTCTCCTCTCAAGCCTCAGCGCTCTCTCTCCC 3′ phosphorylated
Probe 12 (synthetic target for RNA 1 generation)
5′ CCTCCTCTAGTTGGCATGCTTTGATGACGCTTCTGTATCTATATTCATCATAGG
AAACACCAAAGATGATATTTTCTTTAATGGTGCCAGGCATAATCCAGGAAAACT
GAGAACAGAATGA 3′

### Example 6

This example demonstrates that a lateral flow device will detect amplicon from a Nucleic Acid Sequence Based Amplification (NASBA) reaction using Chlamydia trachomatis 16S rRNA as target.

Preparation of Oligonucleotides

All oligonucleotide probes are synthesised and purified as described in the preceding examples.

Preparation of Purified RNA Target Material

A 180 bp 16S rRNA fragment from C. trachomatis is cloned into pGEM-T vector, transcribed and the resulting 16S rRNA fragment transcript purified essentially as described in Song et al. (Combinatorial Chemistry & High Throughput Screening 3, (2000))

Preparation of Lateral Flow Devices

Dilute anti-HRP antibody (Sigma) to 1 mg ml⁻¹ in phosphate buffered saline (PBS) striping buffer. Stripe onto Millipore HF135 nitrocellulose matrix card using a Kinematic Matrix 1600 (Kinematic Automation) stripe width 1.5 μl cm⁻¹, 1 cm from distal end. Dry at 37° C. for 2 hours in an incubator. Add 2 cm Ahlstrom 222 matrix upper wick to the distal end of the Millipore HF135 card to give a 5 mm overlap with the HF135 nitrocellulose. Cut card into 5 mm width dipsticks using a Kinematic Matrix 2360 (Kinematic Automation). Prepare reaction pads by cutting 10 mm strip of Ahlstrom 8964 and cut further into 3 mm width reaction pads using a Kinematic Matrix 2360 (Kinematic Automation). Reaction pads are adhered to Mylar® backing material at the proximal end of the striped 5 mm Millipore HF135 dipsticks to give a 10 mm gap between the reaction pad and the proximal end of the HF135 nitrocellulose (i.e. reaction pad and HF135 nitrocellulose are separated by a 10 mm gap).

NASBA Reaction

The following constituents are placed into two 0.2 ml reaction tubes; 5 μl of purified C. trachomatis 16S rRNA transcript in one tube and 5 μl of dH₂O into the second tube (negative control), 2 μl of enzyme mix and 15 μl amplification mix consisting of 40 mM Tris-HCl pH 8.46, 2 mM each NTP, 1 mM each dNTP, 10 mM DTT, 12 mM MgCl₂, 90 mM KCl, 0.2 μM of each primer (P1 & P2) and 15% DMSO. Enzyme mix containing 40 units T7 polymerase (USB), 8 units AMV-RT (Seikagaka America Inc.), 0.2 units RNase H (USB), 12.5 units RNAguard (Amersham Pharmacia Biotech) and 100 mg ml⁻¹ BSA (Roche Molecular Biochemicals) are added to the reaction. Reaction made up to a final volume of 25 μl with dH₂O. The reaction constituents are transferred from the reaction tube to the reaction pad on the dipstick, covered with parafilm and sealed to prevent evaporation (in absence of dedicated assay device housing) and placed in a 41° C. incubator for 90 minutes.

Development

Prepare 20 mm×5 mm Ahlstrom 8964 matrix strips. Dilute anti-biotin 40 nm gold conjugate (British BioCell International, Cardiff, UK) in Tris Buffered Saline (2.42 g Tris(hydroxymethyl)methylamine, 9.0 g NaCl, 1.3 g sodium azide, 10.0 g BSA, 11.0 g Tween 20, pH8.2 per litre) to 1OD. Add 1 μl 3′ HRP capture probe and 1 μl of biotinylated detection probe to 98 μl of OD 1 anti-biotin conjugate.

After incubation dipsticks are removed from incubator, parafilm removed and 20 mm×5 mm Ahlstrom strip placed to bridge 10 mm gap between reaction pad and nitrocellulose (HF135). 100 μl conjugate/detection/capture probe mix is slowly dispensed onto reaction pad and allowed to migrate along the dipstick for 30 minutes to develop.

Result

A red line indicates the presence of C. trachomatis 16S rRNA transcript. The negative control device does not produce a red line.

List of Oligonucleotides

P1
5′
AATTCTAATACGACTCACTATAGGGAGCACATAGACTCTCCCTTAA 3′
(Underlined sequence indicates the T7 RNA polymer-
ase promoter sequence)
P2
5′ AGCAATTGTTTCGACGATT 3′
Capture Probe
5′ Bio-GGCGGAAGGGTTAGTAATG 3′
Detection Probe
5′ GTGGCGATATTTGGGCATCCGAGTA-HRP 3′

### Example 7

This example demonstrates that a lateral flow device will detect RNA amplicon from a Signal Mediated Amplification of RNA Technology (SMART) reaction using 16S rRNA derived from Salmonella typhimurium as target. Lysis occurs outside of the device, and annealing and amplification occurs on a reaction pad at the proximal end of the device.

Preparation of Oligonucleotides

All oligonucleotide probes were synthesised and purified as described in the preceding examples.

Preparation of Lateral Flow Devices

Anti-HRP antibody (Sigma) was diluted to 1 mg ml⁻¹ in phosphate buffered saline (1.48 g Na₂HPO₄ 0.43 g, KH₂HPO_{4,} 17.2 g NaCl, 1.3 g sodium azide, pH 7.2 per litre) striping buffer and striped onto Millipore HF135 nitrocellulose matrix card using a Kinematic Matrix 1600, stripe width 1.5 μlcm⁻¹, 1 cm from distal end. The card was then dried at 37° C. for 2 hours in an incubator. A 2 cm upper wick (Ahlstrom 222) was then applied to the distal end of the striped Millipore HF135 card to give a 5 mm overlap with the HF135 nitrocellulose. Excess backing card was removed by use of a scalpel. The card was cut into 5 mm width dipsticks using a Kinematic Matrix 2360.

Reaction pads were prepared by cutting a 10 mm strip of Whatman Rapid 24Q followed by adhesion to Millipore HF000MC100 laminated backing card. Excess backing card was removed by use of a scalpel. 5 mm width sample pads were cut using a Kinematic Matrix 2360. Final pad size 10×5 mm. Reaction pads were adhered to Riverside foil backing material via Scotch Double-Sided Artist Tape. The device was completed by the application of the striped 5 mm Millipore HF135 dipsticks 10 mm proximal to the reaction pad to give a 10 mm gap between the reaction pad and the proximal end of the HF135 nitrocellulose (i.e. reaction pad and HF135 nitrocellulose are separated by a 10 mm gap).

Bridge pads were prepared by cutting a 20 mm strip of Ahlstrom 8964 into further 5 mm width reaction pads using a Kinematic Matrix 2360.

Preparation of Target

Salmonella typhimurium ATCC 14028 (positive sample) and Escherichia coli ATCC 25922 (negative sample) were grown overnight in 10 ml of buffered peptone water (Merck). Bacteria were then heat killed at 95° C. for 15 minutes.

SMART Reaction

SMART reaction constituents were added to a 0.2 ml reaction tube to a final concentration in 20 μl: 4 mM rNTP mix, 55 μM dNTP mix, 0.9 pmol Probe 8, lyophilisation mix (sucrose 2.5% w/v, ficoll 0.5% w/v, polyvinylpyrollidone 0.5% w/v), 150 mM NaCl, 100 ng Sigma Micrococcus DNA, 1× Ambion transcription buffer, 10 fmol probe 1, 20 fmol probe 2, 2 pmol probe 3, 2 pmol probe 4, 150 fmol probe 5, 600 fmol probe 6, 6 pmol probe 7 (see “list of oligonucleotides”).

10 μl of target was added prior to transferring the 30 μl reaction to the reaction pad. Devices were incubated at 41° C./30 minutes in a standard laboratory incubator.

SMART reaction enzyme constituents were added to the reaction pad at a final concentration in 60 μl: 800 U Ambion T7 RNA Polymerase, 16 U New England Biolabs Bst DNA Polymerase, 1.5× Ambion Transcription Buffer, lyophilisation mix (sucrose 17.9% w/v, ficoll 3.6% w/v, polyvinylpyrollidone 3.6% w/v), 2 pmol Probe 9. Reaction pads were sealed with 25×15 mm Pechiney parafilm. Devices were incubated at 41° C. for 2 hours in a standard laboratory incubator. 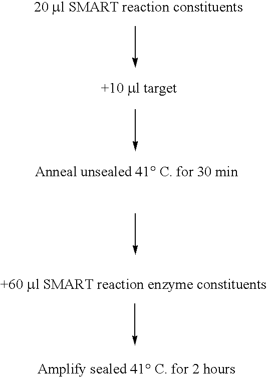
Lateral Flow Assay

The parafilm seal was removed from the device and an Ahlstrom 8964 bridge added to give a 5 mm overlap with the proximal ends of the HF135 nitrocellulose and the Rapid 24Q reaction pad. 100 μl of British Biocell International Anti-Biotin Immunogold Conjugate (BA.Mab40; diluted to 1 OD_{520 nm} in Tris buffered saline (1 litre: 2.42 g Tris(hydroxymethyl)methylamine, 9.0 g NaCl, 1.3 g sodium azide, 10.0 g BSA, 11.0 g Tween 20, pH8.2)) was applied to the reaction pad. A positive result was denoted by the presence of a red line at the anti-HRP stripe position after lateral flow had proceeded for 30 minutes. Lateral flow was stopped by removing the upper wick and bridge.

FIG. 7A shows the components used to perform the assay and FIG. 7B shows a picture of the assembled components following completion of the assay. A coloured band in the detection zone is clearly visible in the positive samples and is not observed in the negative controls.

List of Oligonucleotides for Example 7

Probe 1 (Template)
5′ TCGTCTTCCGGTCTCTCCTCTCAAGCCTCAGCGCTCTCTCTCCCT
ATAGTGAGTCGTATTAATTTCGAA-(octanediol)
TCCCCGCTGAAAGTACTTTACAACCCGAAG-3′ blocker
Probe 2 (Extension)
5′ TATTAACCACAACACCTTCCTTCGAAAT 3′
Probe 3 (Facilitator)
5′ GTAACGTCAATTGCTGCGGT 3′ blocker
Probe 4 (Facilitator)
5′ GCCTTCTTCATACACGCGGC 3′ blocker
Probe 5 (Template)
5′ TGCCTGCTTGTCTGCGTTCTGGATATCACCCGAGCT
CTCTCTCCCTATAGTGAGTCGTATTAATTTCGAA-(Octandiol)
CTCCTCTCAAGCCTC 3′
Probe 6 (Extension)
5′ TCGTCTTCCGGTCTTTCGAAAT 3′
Probe 7 (Facilitator)
5′ AGCGCTCTCTCTCCC 3′
Probe 8 (Biotinylated Probe)
5′ BiotinTCTGCTGCCTGCTTGTCTGCGTTCT 3′
Probe 9 (HRP Probe)
5′ GGATATCACCCG HRP 3′

### Example 8

This example demonstrates that a lateral flow device will detect RNA amplicon from a Signal Mediated Amplification of RNA Technology (SMART) reaction using 16S rRNA derived from Salmonella typhimurium as target. Lysis occurs on a lysis pad at the proximal end of the device, and annealing and amplification occurs on a separate reaction pad.

Preparation of Oligonucleotides

All oligonucleotide probes were synthesised and purified as described in the preceding examples.

Preparation of Lateral Flow Devices

Anti-HRP antibody (Sigma) was diluted to 1 mg ml⁻¹ in phosphate buffered saline (1.48 g Na₂HPO₄ 0.43 g, KH₂HPO_{4,} 17.2 g NaCl, 1.3 g sodium azide, pH 7.2 per litre) striping buffer and striped onto Millipore HF135 nitrocellulose matrix card using a Kinematic Matrix 1600, stripe width 1.5 μl cm⁻¹, 1 cm from distal end. The card was then dried at 37° C. for 2 hours in an incubator. A 2 cm upper wick (Ahlstrom 222) was then applied to the distal end of the striped Millipore HF135 card to give a 5 mm overlap with the HF135 nitrocellulose. Excess backing card was removed by use of a scalpel. The card was cut into 5 mm width dipsticks using a Kinematic Matrix 2360.

Devices were prepared by cutting a 10 mm strip of Whatman FTA Classic Card (Whatman, Maidstone, UK) for the lysis pad, followed by adhesion to a strip of Millipore HF000MC100 backing card. A 10 mm strip of Whatman Rapid 24Q (reaction pad) was adhered to the card to give a 5 mm gap between the lysis and reaction pads. Excess backing card was removed by use of a scalpel. The combined strip was cut into 25×5 mm sections using a Kinematic Matrix 2360. Final lysis pad size 10×5 mm. Final reaction pad size 10×5 mm. Sections were adhered to Riverside foil backing material via Scotch Double-Sided Artist Tape. The device was completed by the application of the striped 5 mm Millipore HF135 dipsticks 10 mm proximal to the reaction pad to give a 10 mm gap between the reaction pad and the proximal end of the HF135 nitrocellulose (i.e. Reaction pad and HF135 nitrocellulose are separated by a 10 mm gap).

Lysis-reaction pad bridges were prepared by cutting a 15 mm strip of Ahlstrom 8964 into further 5 mm width reaction pads using a Kinematic Matrix 2360.

Reaction-nitrocellulose pad bridges were prepared by cutting a 20 mm strip of Ahlstrom 8964 into further 5 mm width reaction pads using a Kinematic Matrix 2360.

Preparation of Target

Salmonella typhimurium ATCC 14028 (positive sample) and Escherichia coli ATCC 25922 (negative sample) were grown overnight in 10 ml of buffered peptone water (Merck). Bacteria were then heat killed at 95° C. for 15 minutes.

SMART Reaction

10 μl of target was added to the lysis pad and incubated at room temperature for 5 min. A lysis-reaction pad bridge was applied to give a 5 mm overlap at each end. 50 μl of TE was applied to the lysis pad and the target wicked to the reaction pad at room temperature for 10 minutes.

SMART reaction constituents were added to the reaction pad to a final concentration in 20 μl: 4 mM rNTP mix, 55 μM dNTP mix, 0.9 pmol probe 8, lyophilisation mix (sucrose 2.5% w/v, ficoll 0.5% w/v, polyvinylpyrollidone 0.5% w/v), 150 mM NaCl, 100 ng Sigma Micrococcus DNA, 1× Ambion transcription buffer, 10 fmol probe 1, 20 fmol probe 2, 2 pmol probe 3, 2 pmol probe 4, 150 fmol probe 5, 600 fmol probe 6, 6 pmol probe 7. Devices were incubated at 41° C. for 60 minutes in a standard laboratory incubator.

Lysis-reaction pad bridges were removed and SMART reaction enzyme constituents added to the reaction pad at a final concentration in 60 μl: 800 U Ambion T7 RNA Polymerase, 16 U New England Biolabs Bst DNA Polymerase, 1.5× Ambion Transcription Buffer, lyophilisation mix (sucrose 17.9% w/v, ficoll 3.6% w/v, polyvinylpyrollidone 3.6% w/v), 2 pmol probe 9. Lysis/Reaction pad sections were sealed with 30×20 mm Pechiney parafilm. Devices were incubated at 41° C. for 2 hours in a standard laboratory incubator. 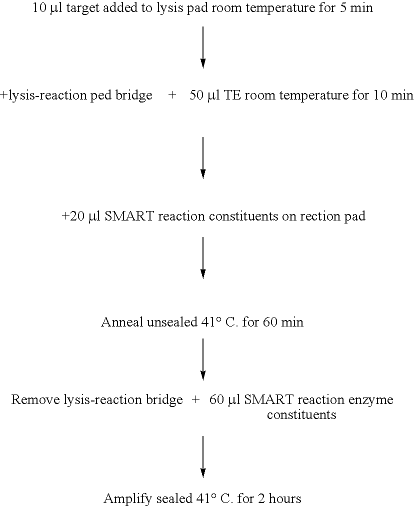
Lateral Flow Assay

An Ahlstrom 8964 bridge was added to the device to give a 5 mm overlap with the proximal ends of the HF135 nitrocellulose and the Rapid 24Q reaction pad.

100 μl of British Biocell International Anti-Biotin Immunogold Conjugate (BA.Mab40) diluted to 1 OD_{520 nm} in Tris buffered saline ((1 litre: 2.42 g Tris(hydroxymethyl)methylamine, 9.0 g NaCl, 1.3 g sodium azide, 10.0 g BSA, 11.0 g Tween 20, pH8.2)) was applied to the reaction pad. A positive result was denoted by the presence of a red line at the anti-HRP stripe position after lateral flow had proceeded for 30 minutes. Lateral flow was stopped by removing the upper wick and bridge.

List of Oligonucleotides for Example 8

Probe 1 (Template)
5′ TCGTCTTCCGGTCTCTCCTCTCAAGCCTCAGCGCTCTCTCTCCCT
ATAGTGAGTCGTATTAATTTCGAA-(octanediol)
TCCCCGCTGAAAGTACTTTACAACCCGAAG-3′ blocker
Probe 2 (Extension)
5′ TATTAACCACAACACCTTCCTTCGAAAT 3′
Probe 3 (Facilitator)
5′ GTAACGTCAATTGCTGCGGT 3′ blocker
Probe 4 (Facilitator)
5′ GCCTTCTTCATACACGCGGC 3′ blocker
Probe 5 (Template)
5′ TGCCTGCTTGTCTGCGTTCTGGATATCACCCGAGCT
CTCTCTCCCTATAGTGAGTCGTATTAATTTCGAA-(Octanediol)
CTCCTCTCAAGCCTC 3′
Probe 6 (Extension)
5′ TCGTCTTCCGGTCTTTCGAAAT 3′
Probe 7 (Facilitator)
5′ AGCGCTCTCTCTCCC 3′
Probe 8 (Biotinylated Probe)
5′ BiotinTCTGCTGCCTGCTTGTCTGCGTTCT 3′
Probe 9 (HRP Probe)
5′ GGATATCACCCG HRP 3′

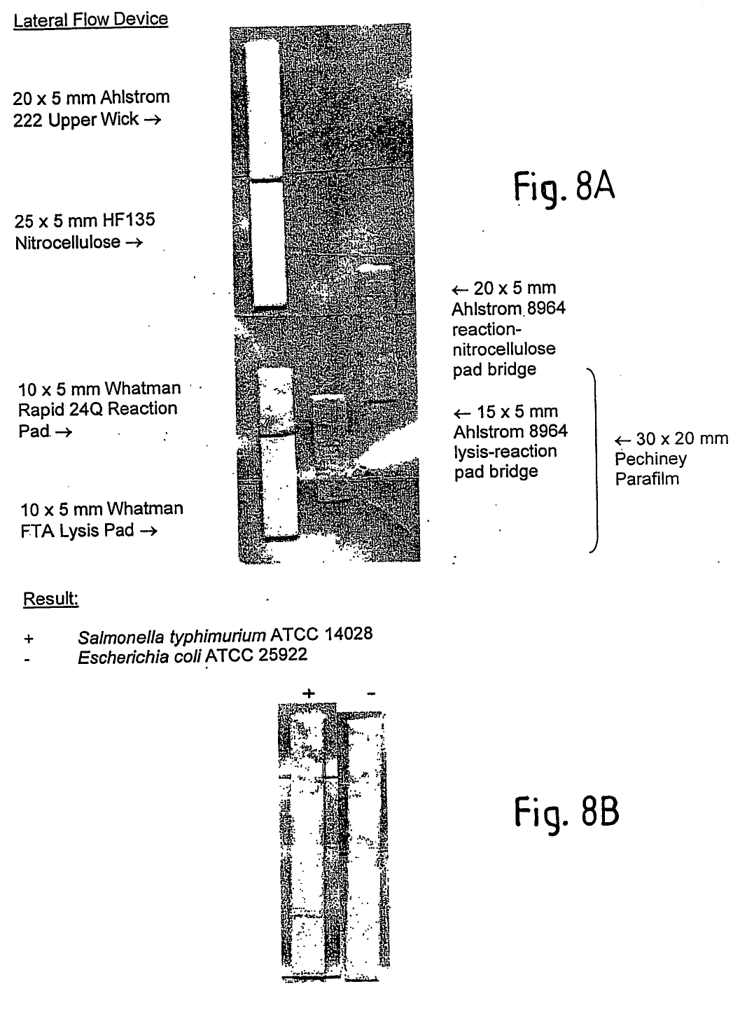

FIG. 8A shows the components used to perform the assay and FIG. 8B shows a picture of the assembled components at the completion of the assay. A coloured band in the detection zone is visible in the positive samples and is not observed in the negative controls.

### Example 9

This example demonstrates that a lateral flow device will detect RNA amplicon from a Signal Mediated Amplification of RNA Technology (SMART) reaction using 16S rRNA derived from Salmonella typhimurium as target. Lysis, annealing and amplification occurred on a combined lysis/amplification pad at the proximal end of the device.

Preparation of Oligonucleotides

All oligonucleotide probes were synthesised and purified as described in preceding examples.

Preparation of Lateral Flow Devices

Anti-HRP antibody (Sigma) was diluted to 1 mg ml⁻¹ in phosphate buffered saline (1.48 g Na₂HPO₄ 0.43 g, KH₂HPO_{4,} 17.2 g NaCl, 1.3 g sodium azide, pH 7.2 per litre) striping buffer and striped onto Millipore HF135 nitrocellulose matrix card using a Kinematic Matrix 1600, stripe width 1.5 μl cm⁻¹, 1 cm from distal end. The card was then dried at 37° C. for 2 hours in an incubator. A 2 cm upper wick (Ahlstrom 222) was then applied to the distal end of the striped Millipore HF135 card to give a 5 mm overlap with the HF135 nitrocellulose. Excess backing card was removed by use of a scalpel. The card was cut into 5 mm width dipsticks using a Kinematic Matrix 2360.

Devices were prepared by cutting a 10 mm strip of Whatman Rapid 24Q (lysis pad) pre-treated with 0.2% (w/v) dodecyl trimethyl ammonium bromide extractant (DTAB; Sigma; D8638). Post-drying (41° C. for 1 hour) pre-treated Whatman Rapid 24Q was adhered to a strip of Millipore HF000MC100 backing card. Excess backing card was removed by use of a scalpel. The combined strip was cut into 10×5 mm sections using a Kinematic Matrix 2360 (final lysis/reaction pad size 10×5 mm). Sections were adhered to Riverside foil backing material via Scotch Double-Sided Artist Tape. The device was completed by the application of the striped 5 mm Millipore HF135 dipsticks 10 mm proximal to the reaction pad to give a 10 mm gap between the reaction pad and the proximal end of the HF135 nitrocellulose (i.e. Reaction pad and HF135 nitrocellulose are separated by a 10 mm gap).

Lysis-reaction pad bridges were prepared by cutting a 15 mm strip of Ahlstrom 8964 into further 5 mm width reaction pads using a Kinematic Matrix 2360. Reaction-nitrocellulose pad bridges were prepared by cutting a 20 mm strip of Ahlstrom 8964 into further 5 mm width reaction pads using a Kinematic Matrix 2360.

Preparation of Target

Salmonella typhimurium ATCC 14028 (positive sample) and Escherichia coli ATCC 25922 (negative sample) were grown overnight in 10 ml of buffered peptone water (Merck). Bacteria were then heat killed at 95° C. for 15 minutes.

SMART Reaction

10 μl of target was added to the combined lysis/amplification pad. DTAB extractant was immediately neutralized by the addition of SMART reaction constituents to a final concentration in 20 μl: 4 mM rNTP mix, 55 μM dNTP mix, 0.9 pmol Probe 8, lyophilisation mix (sucrose 2.5% w/v, ficoll 0.5% w/v, polyvinylpyrollidone 0.5% w/v), 150 mM NaCl, 100 ng Sigma Micrococcus DNA, 1× Ambion transcription buffer, 10 fmol probe 1, 20 fmol probe 2, 2 pmol probe 3, 2 pmol probe 4, 150 fmol probe 5, 600 fmol probe 6, 6 pmol probe 7, 1.44% (w/v) α-cyclodextrin (Sigma, C4642). The cyclodextrin neutralises the DTAB, which would otherwise inhibit the reaction enzymes. Devices were incubated at 41° C./30 minutes in a standard laboratory incubator.

SMART reaction enzyme constituents were added to the reaction pad at a final concentration in 60 μl: 800 U Ambion T7 RNA Polymerase, 16 U New England Biolabs Bst DNA Polymerase, 1.5× Ambion Transcription Buffer, lyophilisation mix (sucrose 17.9% w/v, ficoll 3.6% w/v, polyvinylpyrollidone 3.6% w/v), 2 pmol Probe 9. Lysis/Reaction pad sections were sealed with 30×20 mm Pechiney parafilm. Devices were incubated at 41° C. for 2 hours in a standard laboratory incubator. 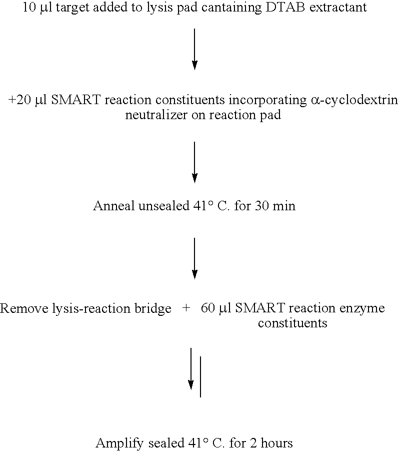
Lateral Flow Assay

An Ahlstrom 8964 bridge was added to the device to give a 5 mm overlap with the proximal ends of the HF135 nitrocellulose and the Rapid 24Q reaction pad.

100 μl of British Biocell International Anti-Biotin Immunogold Conjugate (BA.Mab40; diluted to 1 OD_{520 nm} in Tris buffered saline ((1 litre: 2.42 g Tris(hydroxymethyl)methylamine, 9.0 g NaCl, 1.3 g sodium azide, 10.0 g BSA, 11.0 g Tween 20, pH8.2)) was applied to the reaction pad. A positive result was denoted by the presence of a red line at the anti-HRP stripe position after lateral flow had proceeded for 30 minutes. Lateral flow was stopped by removing the upper wick and bridge.

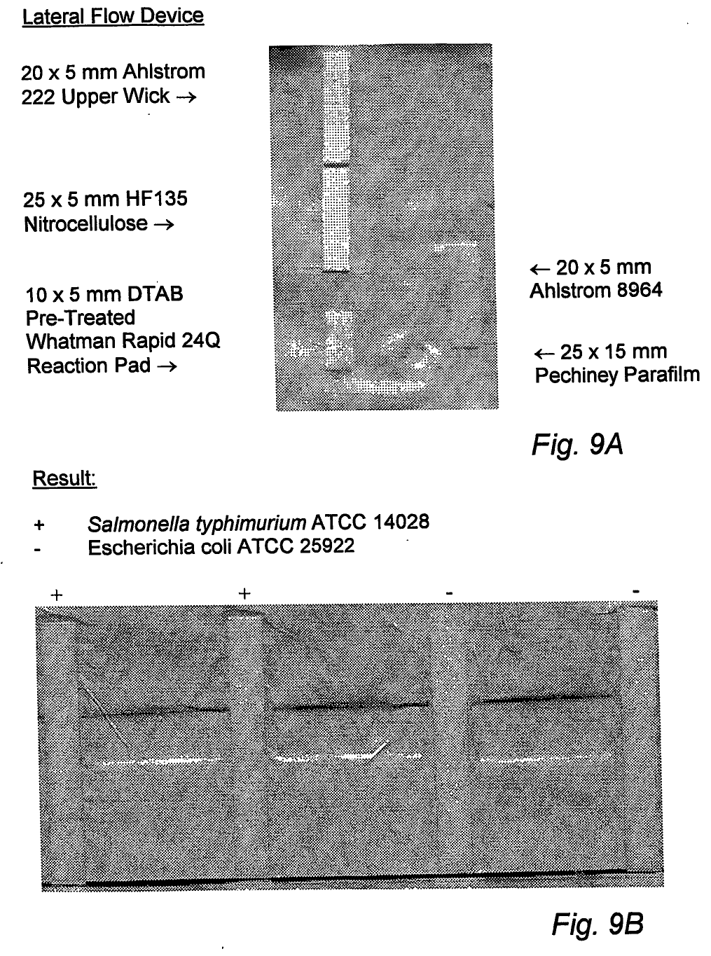

FIG. 9A shows the components used to perform the assay and FIG. 9B shows a picture of the assembled components at the completion of the assay. A coloured band in the detection zone is visible in the positive samples and is not observed in the negative controls.

List of Oligonucleotides

Probe 1 (Template)
5′ TCGTCTTCCGGTCTCTCCTCTCAAGCCTCAGCGCTCTCTCTCCCT
ATAGTGAGTCGTATTAATTTCGAA-(octanediol)
TCCCCGCTGAAAGTACTTTACAACCCGAAG-3′ blocker
Probe 2 (Extension)
5′ TATTAACCACAACACCTTCCTTCGAAAT 3′
Probe 3 (Facilitator)
5′ GTAACGTCAATTGCTGCGGT 3′ blocker
Probe 4 (Facilitator)
5′ GCCTTCTTCATACACGCGGC 3′ blocker
Probe 5 (Template)
5′ TGCCTGCTTGTCTGCGTTCTGGATATCACCCGAGCT
CTCTCTCCCTATAGTGAGTCGTATTAATTTCGAA-(Octanediol)
CTCCTCTCAAGCCTC 3′ blocker
Probe 6 (Extension)
5′ TCGTCTTCCGGTCTTTCGAAAT 3′
Probe 7 (Facilitator)
5′ AGCGCTCTCTCTCCC 3′ blocker
Probe 8 (Biotinylated Probe)
5′ BiotinTCTGCTGCCTGCTTGTCTGCGTTCT 3′
Probe 9 (HRP Probe)
5′ GGATATCACCCG HRP 3′

### Example 10

Referring to FIG. 1, an assay device in accordance with both the first and second aspects of the invention comprises a lateral flow assay strip, indicated generally by reference numeral 1. The strip is provided with a backing of clear synthetic plastics material, such as Mylar® sheet.

The strip is substantially enclosed within a casing of opaque plastics material, forming a protective casing 2 (denoted by the broken line). The casing has an aperture 4 at a proximal, upstream end of the device and a window 6 towards the distal, downstream end. The aperture 4 allows the lateral flow strip to project beyond the casing, at which proximal end there is a sample receiving zone 8. The window 6 allows a user to observe the formation of a test result signal at the test line 10 and a control result signal at the control line 12. The sample receiving zone 8 comprises Whatman FTA paper, which material is useful for performing an extraction step, so that the sample receiving zone 8 is in effect a combined sample receiving and extraction zone.

The sample receiving zone 8 is in liquid flow communication or contact with a porous matrix denoted generally by reference numeral 14, which is itself in liquid flow contact with a wicking member 16 of highly absorbent material (e.g. Ahlstrom 222 in a pad of dimensions approximately 5 mm by 20 mm).

Adjacent to, and slightly overlapping with, the combined sample receiving and extraction zone 8 is an amplification zone 18 which comprises a pad of Whatman GF/C porous material comprising reagents for performing an isothermal SMART nucleic acid amplification, the reagents comprising:

(i) Template probe attached to 2 μm latex microparticles (amine-modified and crosslinked via phenyldiisothiocyanate);

(ii) Extension probe; (iii) DNA polymerase; (iv) RNA polymerase; (v) dNTPs; (vi) rNTPs; (vii) Linear amplification probe; and (viii) amplicon-specific labelling probe coupled to 40 nm gold colloid, prepared by incubation of 40 nm gold colloid (British BioCell) with thiol-capped probe for 1 hour, following by blocking excess binding sites with 1 mg ml⁻¹ BSA.

A mixture containing (i)-(viii) at the appropriate concentrations is prepared in transcription buffer comprising 160 mM Tris (pH 7.8), 24 mM MgCl₂, 8 mM spermidine, 40 mM DTT, 600 mM NaCl, 0.002% Micrococcus DNA, 1% Ficoll and 1% PVP, also containing 5% w/v sucrose, and 50 μl dispensed onto the pad. The pad is then dried by lyophilisation.

The amplification zone 18 is adjacent to, and slightly overlapping with, detection zone 20. The overlap ensures good liquid flow communication between the respective zones of the porous matrix 14. The detection zone 20 comprises a strip of nitrocellulose (HF 135, Millipore) 5 mm×25 mm. Immobilized on the nitrocellulose at test line 10 is an amplicon-specific capture molecule, in the form of a probe oligonucleotide complementary to the sequence of the amplicon. The test line 10 is formed by suspending the amplicon-specific capture probe in 25 mM phosphate buffer (pH 7.0) containing 0.5 mg/ml BSA, and depositing a stripe of the suspension across the nitrocellulose, which is then dried overnight at 21° C. at a relative humidity of less than 20%.

The control line 12 may be formed in a substantially similar manner, using a capture molecule specific for the labelling probe.

The combined sample receiving and extraction zone 8, amplification zone 18, and detection zone 20 are laminated onto adhesive-backed Mylar sheet (from Adhesives Research) to provide support and ensure their correct orientation. Liquid flow between the components is ensured by providing a 2 mm overlap between adjacent components. The components 8, 18 and 20, with their Mylar backing are placed within a moulded synthetics plastics material which forms protective casing 2. Internal projections within the casing 2 at the points of overlap ensure good liquid flow communication between adjacent components.

A large number of variants of the illustrated embodiment can be readily envisaged e.g. the use of a moiety, such as a nucleic acid probe (especially a SMART assay template probe) bound to labelled latex particles which are deposited in dry form on the porous matrix of the assay device and which are mobilised on contact with a carrier liquid and hence migrate along the assay device whereupon they may be captured by a capture moiety deposited on a control line which has specific binding activity for a moiety on the template probe or the latex particle on which it is supported, thereby forming a visible control result signal at the control line, providing a visible indication to the test user that sufficient liquid has been contacted with the sample receiving zone to mobilise the reagent(s) releasably bound to the porous matrix.

### Example 11

Assay for E. coli 23S rRNA

This example relates to an assay device and method in accordance with the invention, for the detection of E. coli 23S rRNA. The apparatus is illustrated schematically in FIG. 2, in longitudinal section. Components of the illustrated apparatus analogous to the embodiment represented in FIG. 1 are denoted by the same reference numerals.

As before, a combined sample receiving and extraction zone 8 (comprising FTA paper), an amplification zone 18, a detection zone 20 and a wicking member 16 are laminated onto a piece of adhesive-backed Mylar® 22 and substantially enclosed within a moulded plastics coating (not shown).

At the junction of the amplification zone 18 and the detection zone 20, a 2 mm gap is left between the portions which are adhered to the Mylar® backing 20. A non-adhered flap 24 of the amplification zone 18 is provided, 5 mm in length. The flap 24 overlaps the detection zone 20 but liquid flow communication between the amplification zone 18 and the detection zone 20 is initially prevented by the presence of an intervening removable sheet 26 of impermeable plastics material, which at least partially projects through an aperture provided in the casing. The aperture may be the same as result window 6 (in FIG. 1) or be a separate aperture.

To perform an assay, sample is added onto the sample receiving and extraction zone 8 and the device placed on a heated block at 41° C. for lysis and release of nucleic acids. Carrier fluid (e.g. TE buffer) is then added from a dropper bottle or micro pipette, causing the extracted nucleic acid to migrate by capillary action to the amplification zone 18 and mobilise the reagents releasably bound therein. Typically between 50 μl to 2 ml of carrier fluid is added, preferably between 100 μl and 1 ml.

Following the mobilisation of the amplification reagents within the amplification zone 18 by the carrier fluid containing the released nucleic acid, generation of amplicon ensues if the target (23S rRNA from E. coli) sequence is present. As the amplicon is generated by the amplification reaction, it binds to a colloidal gold-labelled amplicon-specific probe to form a labelled amplicon/amplicon-specific probe complex.

Because the liquid flow communication with the detection zone 20 is blocked by the removable plastics, sheet 26, the amplicon generated, and the resulting labelled complex, accumulate in the amplification zone 18.

After 40 mins, the plastics separation sheet 26 is removed, allowing liquid, together with labelled amplicon and any excess free labelled amplicon detection probe to migrate into the detection zone 20. Conveniently a projection and/or biasing member is provided on the inner surface of the casing, to urge the amplification zone 18 into intimate contact with the detection zone 20 once the impermeable plastics sheet 26 is removed. Any labelled amplicon present becomes bound to the amplicon detection probe immobilised at the test line 10 and forms a red-coloured line. The free labelled amplicon detection probe migrates past the test line and is captured by a probe-specific capture moiety immobilised at the control line 12, forming a visible control result.

The test and control lines can be visualized through a window in the casing (or quantified by a reader), and a red line is indicative of presence of E. coli 23S rRNA target in the sample.

### Example 12

A further embodiment is illustrated schematically in FIG. 3. Again, components of the assay device which are analogous to those shown in FIG. 2 are denoted by common reference numerals.

In this embodiment there is provided an air gap between the amplification zone 18 and the detection zone 20. After an appropriate amount of time has elapsed, to allow amplicon to accumulate in the amplification zone 18, the relative positions of at least part of the amplification zone 18 and the detection zone 20 are altered to establish liquid flow communication therebetween. In the particular embodiment illustrated in FIG. 3, the positions of at least a flap part 24 of the amplification zone 18 is altered, relative to the detection zone 20, by actuation of a plunger or push-button 28 which is received within an aperture 30 provided in the casing 2. Depression of the plunger or push-button 28 causes the component to bear down on the flap 24, pushing it into intimate contact with the detection zone 20, thereby allowing liquid and any accumulated amplicon and other mobilised substances to pass into the detection zone 20.

It should be noted that in the embodiments illustrated in FIGS. 2 and 3, interruption of the liquid flow path between the amplification zone 18 and the detection zone 20 also has the result of removing the wicking effect of wicking member 16. It is important therefore that the amplification zone 18 is of reasonable absorbency to provide sufficient capillary flow to draw analyte and/or reagents from the sample receiving/extraction zone 8.

1. A lateral flow assay device to test for the presence and/or amount of a nucleic acid sequence of interest in a sample comprising:
(a) a sample receiving zone for contacting the device with a sample to be tested;
(b) an extraction zone for extraction of nucleic acid from the sample;
(c) a nucleic acid amplification zone in liquid communication with the sample receiving zone; and
(d) a detection zone for detecting the product/s, directly or indirectly, of a nucleic acid amplification reaction performed in the nucleic acid amplification zone, said detection zone being in liquid communication with the amplification zone; the device also comprising a porous matrix which, at a proximal end, is in liquid communication with the sample receiving zone such that liquid applied to the sample receiving zone flows along the device through the porous matrix by capillary action.

2. The lateral flow assay device according to claim 1, wherein the nucleic acid amplification comprises an isothermal amplification reaction.

3-26. (canceled)

27. The lateral flow assay device according to claim 1, wherein the device comprises one or more reagents releasably bound on the porous matrix.

28. The lateral flow assay device according to claim 27, wherein the one or more reagents releasably bound comprise one or more reagents required to perform the nucleic acid amplification reaction.

29. The lateral flow assay device according to claim 1, comprising one or more reagents immobilized on the porous matrix.

30. The lateral flow assay device according to claim 29, wherein the one or more immobilized reagents comprise an amplification-specific capture moiety.

31. The lateral flow assay device according to claim 1, comprising a probe comprising nucleic acid releasably bound or immobilized on the porous matrix.

32. The lateral flow assay device according to claim 1, wherein the sample receiving zone comprises reagents suitable to perform a nucleic acid extraction step on a sample applied to the sample receiving zone.

33. The lateral flow assay device according to claim 1, comprising dodecyl trimethyl ammonium bromide, FTA paper, or a matrix comprising one or more agents for cell lysis and nucleic acid protection.

34. The lateral flow assay device according to claim 1, comprising means for interruption of flow, alteration of rate of flow, or alteration of flow path, of a liquid along the porous matrix within the device.

35. The lateral flow assay device according to claim 34, comprising means for altering the relative positions of two or more portions of the porous matrix, so as to affect the rate of flow of liquid from one portion to another.

36. The lateral flow assay device according to claim 1, wherein the amplification reaction comprises a SMART amplification reaction involving the sequence of interest in the formation of a three way junction with two probe molecules.

37. An assay kit for performing an assay to test for the presence and/or amount of a nucleic acid sequence of interest in a sample, the kit comprising a lateral flow assay device according to claim 1, and a supply of at least one reagent required to perform the assay.

38. The assay kit according to claim 37, comprising a supply of carrier liquid.

39. The assay kit according to claim 38, wherein at least one reagent is provided dissolved and/or suspended in the carrier liquid.

40. A method of performing an assay to test for the presence and/or amount of a nucleic acid sequence of interest in a sample, comprising: contacting the sample with the sample receiving zone of a lateral flow assay device according to claim 1, so as to cause a nucleic acid amplification reaction in the presence of the sequence of interest; and detecting, directly or indirectly, the product/s of the amplification reaction in the detection zone of the lateral flow assay device.

41. The method according to claim 40, wherein the amplification reaction comprises a SMART amplification reaction involving the sequence of interest in the formation of a three way junction with two probe molecules.

42. The method according to claim 40, wherein the method comprises the step of performing a nucleic acid extraction step in an extraction zone of the assay device.

43. The method according to claim 42, wherein the extraction step comprises contacting nucleic acid in the sample with dodecyl trimethyl ammonium bromide (“DTAB”) and subsequently contacting the extracted nucleic acid/DTAB mixture with cyclodextrin.

44. The method of making a lateral flow assay device according to claim 1 comprising: forming a porous matrix comprising an amplification zone and a detection zone, said amplification zone being in liquid flow communication with a sample receiving zone, the sample receiving zone comprising one or more reagents immobilized or releasably bound thereon so as to perform a nucleic acid extraction step on a nucleic-acid containing sample contacted with the sample receiving zone.
